SEQUENCE LISTING (86 sequences)

SEQ ID NO: 1 - DNA, 537 bp, Homo sapiens
atgtctgggggcaaatacgtagactcggagggacatctctacaccgttcccatccgggaacagggcaacatctacaagcccaacaacaaggccatggcagacgagctgagcgagaagcaagtgtacgacgcgcacaccaaggagatcgacctggtcaaccgcgaccctaaacacctcaacgatgacgtggtcaagattgactttgaagatgtgattgcagaaccagaagggacacacagttttcacggcatttggaaggccagcttcaccaccttcactgtgacgaaatactggttttaccgcttgctgtctgccctctttggcatcccgatggcactcatctggggcatttacttcgccattctctctttcctgcacatctgggcagttgtaccatgcattaagagcttcctgattgagattcagtgcaccagccgtgtctattccatctacgtccacaccgtctgtgacccactctttgaagctgttgggaaaatattcagcaatgtccgcatcaacttgcagaaagaaatataa

SEQ ID NO: 2 - protein, 20 aa, Homo sapiens
HGIWKASFTTFTVTKYWFYR

SEQ ID NO: 3 - protein, 22 aa, Homo sapiens
KSFLIEIQCTSRVYSIYVHTVC

SEQ ID NO: 4 - protein, 10 aa, Artificial Sequence
  Description of Artificial Sequence Caveolin binding motif
FPAAPFSGWY

SEQ ID NO: 5 - DNA, 40 bp, Artificial Sequence
  Description of Artificial Sequence Partial antisense sequence of human caveolin-1
gagtctacgtatttgcccccagacatgctggcccgtggct

SEQ ID NO: 6 - protein, 10 aa, Artificial Sequence
  Description of Artificial Sequence Caveolin binding motif
FXFXXXXFXF

SEQ ID NO: 7 - protein, 10 aa, Artificial Sequence
  Description of Artificial Sequence Caveolin binding motif
FXFXXXXFXY

SEQ ID NO: 8 - protein, 10 aa, Artificial Sequence
  Description of Artificial Sequence Caveolin binding motif
FXFXXXXYXY

SEQ ID NO: 9 - protein, 10 aa, Artificial Sequence
  Description of Artificial Sequence Caveolin binding motif
FXYXXXXYXY

SEQ ID NO: 10 - protein, 10 aa, Artificial Sequence
  Description of Artificial Sequence Caveolin binding motif
FXWXXXXYXY

SEQ ID NO: 11 - protein, 10 aa, Artificial Sequence
  Description of Artificial Sequence Caveolin binding motif
FXFXXXXWXY

SEQ ID NO: 12 - protein, 10 aa, Artificial Sequence
  Description of Artificial Sequence Caveolin binding motif
FXYXXXXWXY

SEQ ID NO: 13 - protein, 10 aa, Artificial Sequence
  Description of Artificial Sequence Caveolin binding motif
FXYXXXXWXY

SEQ ID NO: 14 - protein, 10 aa, Artificial Sequence
  Description of Artificial Sequence Caveolin binding motif
FXYXXXXFXY

SEQ ID NO: 15 - protein, 10 aa, Artificial Sequence
  Description of Artificial Sequence Caveolin binding motif
FXWXXXXFXY

SEQ ID NO: 16 - protein, 10 aa, Artificial Sequence
  Description of Artificial Sequence Caveolin binding motif
FXFXXXXFXW

SEQ ID NO: 17 - protein, 10 aa, Artificial Sequence
  Description of Artificial Sequence Caveolin binding motif
FXFXXXXYXW

SEQ ID NO: 18 - protein, 10 aa, Artificial Sequence
  Description of Artificial Sequence Caveolin binding motif
FXYXXXXYXW

SEQ ID NO: 19 - protein, 10 aa, Artificial Sequence
  Description of Artificial Sequence Caveolin binding motif
FXWXXXXYXW

SEQ ID NO: 20 - protein, 10 aa, Artificial Sequence
  Description of Artificial Sequence Caveolin binding motif
FXFXXXXWXW

SEQ ID NO: 21 - protein, 10 aa, Artificial Sequence
  Description of Artificial Sequence Caveolin binding motif
FXYXXXXWXW

SEQ ID NO: 22 - protein, 10 aa, Artificial Sequence
  Description of Artificial Sequence Caveolin binding motif
FXWXXXXWXW

SEQ ID NO: 23 - protein, 10 aa, Artificial Sequence
  Description of Artificial Sequence Caveolin binding motif
FXYXXXXFXW

SEQ ID NO: 24 - protein, 10 aa, Artificial Sequence
  Description of Artificial Sequence Caveolin binding motif
FXWXXXXFXW

SEQ ID NO: 25 - protein, 10 aa, Artificial Sequence
  Description of Artificial Sequence Caveolin binding motif
FXFXXXXYXF

SEQ ID NO: 26 - protein, 10 aa, Artificial Sequence
  Description of Artificial Sequence Caveolin binding motif
FXYXXXXYXF

SEQ ID NO: 27 - protein, 10 aa, Artificial Sequence
  Description of Artificial Sequence Caveolin binding motif
FXWXXXXYXF

SEQ ID NO: 28 - protein, 10 aa, Artificial Sequence
  Description of Artificial Sequence Caveolin binding motif
FXFXXXXWXF

SEQ ID NO: 29 - protein, 10 aa, Artificial Sequence
  Description of Artificial Sequence Caveolin binding motif
FXYXXXXWXF

SEQ ID NO: 30 - protein, 10 aa, Artificial Sequence
  Description of Artificial Sequence Caveolin binding motif
FXWXXXXWXF

SEQ ID NO: 31 - protein, 10 aa, Artificial Sequence
  Description of Artificial Sequence Caveolin binding motif
FXYXXXXFXF

SEQ ID NO: 32 - protein, 10 aa, Artificial Sequence
  Description of Artificial Sequence Caveolin binding motif
FXWXXXXFXF

SEQ ID NO: 33 - protein, 10 aa, Artificial Sequence
  Description of Artificial Sequence Caveolin binding motif
YXFXXXXFXF

SEQ ID NO: 34 - protein, 10 aa, Artificial Sequence
  Description of Artificial Sequence Caveolin binding motif
YXFXXXXFXY

SEQ ID NO: 35 - protein, 10 aa, Artificial Sequence
  Description of Artificial Sequence Caveolin binding motif
YXFXXXXYXY

SEQ ID NO: 36 - protein, 10 aa, Artificial Sequence
  Description of Artificial Sequence Caveolin binding motif
YXYXXXXYXY

SEQ ID NO: 37 - protein, 10 aa, Artificial Sequence
  Description of Artificial Sequence Caveolin binding motif
YXWXXXXYXY

SEQ ID NO: 38 - protein, 10 aa, Artificial Sequence
  Description of Artificial Sequence Caveolin binding motif
YXFXXXXWXY

SEQ ID NO: 39 - protein, 10 aa, Artificial Sequence
  Description of Artificial Sequence Caveolin binding motif
YXYXXXXWXY

SEQ ID NO: 40 - protein, 10 aa, Artificial Sequence
  Description of Artificial Sequence Caveolin binding motif
YXWXXXXWXY

SEQ ID NO: 41 - protein, 10 aa, Artificial Sequence
  Description of Artificial Sequence Caveolin binding motif
YXFXXXXFXW

SEQ ID NO: 42 - protein, 10 aa, Artificial Sequence
  Description of Artificial Sequence Caveolin binding motif
YXFXXXXYXW

SEQ ID NO: 43 - protein, 10 aa, Artificial Sequence
  Description of Artificial Sequence Caveolin binding motif
YXYXXXXYXW

SEQ ID NO: 44 - protein, 10 aa, Artificial Sequence
  Description of Artificial Sequence Caveolin binding motif
YXWXXXXYXW

SEQ ID NO: 45 - protein, 10 aa, Artificial Sequence
  Description of Artificial Sequence Caveolin binding motif
YXFXXXXWXW

SEQ ID NO: 46 - protein, 10 aa, Artificial Sequence
  Description of Artificial Sequence Caveolin binding motif
YXYXXXXWXW

SEQ ID NO: 47 - protein, 10 aa, Artificial Sequence
  Description of Artificial Sequence Caveolin binding motif
YXWXXXXWXW

SEQ ID NO: 48 - protein, 10 aa, Artificial Sequence
  Description of Artificial Sequence Caveolin binding motif
YXFXXXXYXF

SEQ ID NO: 49 - protein, 10 aa, Artificial Sequence
  Description of Artificial Sequence Caveolin binding motif
YXYXXXXYXF

SEQ ID NO: 50 - protein, 10 aa, Artificial Sequence
  Description of Artificial Sequence Caveolin binding motif
YXWXXXXYXF

SEQ ID NO: 51 - protein, 10 aa, Artificial Sequence
  Description of Artificial Sequence Caveolin binding motif
YXFXXXXWXF

SEQ ID NO: 52 - protein, 10 aa, Artificial Sequence
  Description of Artificial Sequence Caveolin binding motif
YXYXXXXWXF

SEQ ID NO: 53 - protein, 10 aa, Artificial Sequence
  Description of Artificial Sequence Caveolin binding motif
YXWXXXXWXF

SEQ ID NO: 54 - protein, 10 aa, Artificial Sequence
  Description of Artificial Sequence Caveolin binding motif
YXYXXXXFXF

SEQ ID NO: 55 - protein, 10 aa, Artificial Sequence
  Description of Artificial Sequence Caveolin binding motif
YXWXXXXFXF

SEQ ID NO: 56 - protein, 10 aa, Artificial Sequence
  Description of Artificial Sequence Caveolin binding motif
YXYXXXXFXY

SEQ ID NO: 57 - protein, 10 aa, Artificial Sequence
  Description of Artificial Sequence Caveolin binding motif
YXWXXXXFXY

SEQ ID NO: 58 - protein, 10 aa, Artificial Sequence
  Description of Artificial Sequence Caveolin binding motif
YXYXXXXFXW

SEQ ID NO: 59 - protein, 10 aa, Artificial Sequence
  Description of Artificial Sequence Caveolin binding motif
YXWXXXXFXW

SEQ ID NO: 60 - protein, 10 aa, Artificial Sequence
  Description of Artificial Sequence Caveolin binding motif
WXFXXXXFXF

SEQ ID NO: 61 - protein, 10 aa, Artificial Sequence
  Description of Artificial Sequence Caveolin binding motif
WXFXXXXFXY

SEQ ID NO: 62 - protein, 10 aa, Artificial Sequence
  Description of Artificial Sequence Caveolin binding motif
WXFXXXXYXY

SEQ ID NO: 63 - protein, 10 aa, Artificial Sequence
  Description of Artificial Sequence Caveolin binding motif
WXYXXXXYXY

SEQ ID NO: 64 - protein, 10 aa, Artificial Sequence
  Description of Artificial Sequence Caveolin binding motif
WXWXXXXYXY

SEQ ID NO: 65 - protein, 10 aa, Artificial Sequence
  Description of Artificial Sequence Caveolin binding motif
WXFXXXXWXY

SEQ ID NO: 66 - protein, 10 aa, Artificial Sequence
  Description of Artificial Sequence Caveolin binding motif
WXYXXXXWXY

SEQ ID NO: 67 - protein, 10 aa, Artificial Sequence
  Description of Artificial Sequence Caveolin binding motif
WXWXXXXWXY

SEQ ID NO: 68 - protein, 10 aa, Artificial Sequence
  Description of Artificial Sequence Caveolin binding motif
WXFXXXXFXW

SEQ ID NO: 69 - protein, 10 aa, Artificial Sequence
  Description of Artificial Sequence Caveolin binding motif
WXFXXXXYXW

SEQ ID NO: 70 - protein, 10 aa, Artificial Sequence
  Description of Artificial Sequence Caveolin binding motif
WXYXXXXYXW

SEQ ID NO: 71 - protein, 10 aa, Artificial Sequence
  Description of Artificial Sequence Caveolin binding motif
WXWXXXXYXW

SEQ ID NO: 72 - protein, 10 aa, Artificial Sequence
  Description of Artificial Sequence Caveolin binding motif
WXFXXXXWXW

SEQ ID NO: 73 - protein, 10 aa, Artificial Sequence
  Description of Artificial Sequence Caveolin binding motif
WXYXXXXWXW

SEQ ID NO: 74 - protein, 10 aa, Artificial Sequence
  Description of Artificial Sequence Caveolin binding motif
WXWXXXXWXW

SEQ ID NO: 75 - protein, 10 aa, Artificial Sequence
  Description of Artificial Sequence Caveolin binding motif
WXFXXXXYXF

SEQ ID NO: 76 - protein, 10 aa, Artificial Sequence
  Description of Artificial Sequence Caveolin binding motif
WXYXXXXYXF

SEQ ID NO: 77 - protein, 10 aa, Artificial Sequence
  Description of Artificial Sequence Caveolin binding motif
WXWXXXXYXF

SEQ ID NO: 78 - protein, 10 aa, Artificial Sequence
  Description of Artificial Sequence Caveolin binding motif
WXFXXXXWXF

SEQ ID NO: 79 - protein, 10 aa, Artificial Sequence
  Description of Artificial Sequence Caveolin binding motif
WXYXXXXWXF

SEQ ID NO: 80 - protein, 10 aa, Artificial Sequence
  Description of Artificial Sequence Caveolin binding motif
WXWXXXXWXF

SEQ ID NO: 81 - protein, 10 aa, Artificial Sequence
  Description of Artificial Sequence Caveolin binding motif
WXYXXXXFXF

SEQ ID NO: 82 - protein, 10 aa, Artificial Sequence
  Description of Artificial Sequence Caveolin binding motif
WXWXXXXFXF

SEQ ID NO: 83 - protein, 10 aa, Artificial Sequence
  Description of Artificial Sequence Caveolin binding motif
WXYXXXXFXY

SEQ ID NO: 84 - protein, 10 aa, Artificial Sequence
  Description of Artificial Sequence Caveolin binding motif
WXWXXXXFXY

SEQ ID NO: 85 - protein, 10 aa, Artificial Sequence
  Description of Artificial Sequence Caveolin binding motif
WXYXXXXFXW

SEQ ID NO: 86 - protein, 10 aa, Artificial Sequence
  Description of Artificial Sequence Caveolin binding motif
WXWXXXXFXW

(19) United States
(12) Patent Application Publication
(10) Pub. No.: US 2004/0110684 A1
(43) Pub. Date: Jun. 10, 2004

(54) Novel pharmaceutical compositions for modulating angiogenesis

(75) Inventors: Jean-Luc Balligand, Kraainem (BE); Olivier Feron, Wezembeek-Oppem (BE)

Correspondence Address: MILLEN, WHITE, ZELANO & BRANIGAN, P.C., 2200 CLARENDON BLVD., SUITE 1400, ARLINGTON, VA, 22201, US

(73) Assignee: UNIVERSITE CATHOLIQUE DE LOUVAIN, Louvain-la-Neuve (BE)

(21) Appl. No.: 10/651,024

(22) Filed: Aug. 29, 2003

Related U.S. Application Data
(63) Continuation-in-part of application No. 10/068,965, filed on Feb. 11, 2002.
Application No. 10/068,965 is a continuation-in-part of application No. PCT/EP00/07731, filed on Aug. 9, 2000.

(30) Foreign Application Priority Data
Aug. 2, 1999 (EP) EP99870171.8

(51) Int. Cl. A61K 48/00; A61K 38/17

(52) U.S. Cl. 514/12; 514/44

(57) ABSTRACT

The present invention is related to a pharmaceutical composition for modulating angiogenesis comprising a therapeutically effective amount of an angiogenesis modulating compound and a pharmaceutically acceptable excipient, wherein said modulating compound is a recombinant of caveolin-1, a nucleic acid encoding the partial or total amino acid sequence of caveolin-1, or an analogue thereof or a pharmacologically acceptable derivative thereof, a compound modulating the expression of caveolin-1, an agonist or an antagonist or a competitive inhibitor of caveolin-1, a recombinant hsp90, a nucleic acid encoding the partial or total amino acid sequence of hsp90 or an analogue thereof or a pharmacologically acceptable derivative thereof, a compound modulating the expression of hsp90, an agonist or an antagonist or a competitive inhibitor of hsp90, a recombinant of Akt, a nucleic acid encoding the partial or total amino acid sequence of Akt, or an analogue thereof or a pharmacologically acceptable derivative thereof, a compound modulating the expression of Akt, an agonist or an antagonist or a competitive inhibitor of Akt.

This application is a continuation-in-part of U.S. application Ser. No. 10/068,965 filed Feb. 11, 2002 which is a continuation-in-part of international application PCT/EP00/07731 filed on Aug. 9, 2000, and claims benefit of priority also to European application EP99870171.8 filed on Aug. 9, 1999, the entirety of the disclosures of all of which are fully incorporated by reference herein.

### FIELD OF THE INVENTION

The present invention is related to a pharmaceutical composition for the modulation of angiogenesis, more in particular, for the prevention and/or the treatment of various diseases and pathologies of mammals, including of human, such as ischemic heart and peripheral vascular including cerebral diseases and tumour development and for wound healing.

### TECHNOLOGICAL BACKGROUND OF THE INVENTION

The production of blood vessels, or angiogenesis, is of main importance in biology as blood vessels are the main route to provide food and other essential elements to cells when present in a complex multicellular organism. Its is known that the decrease of the blood stream in said vessels may lead to the deprivation of these essential elements, resulting in the starvation or even in the killing of said cells. Many diseases result from such kind of deprivation. Therefore it is essential to find some tools enabling the modulation of angiogensis.

A therapeutic angiogenesis favour the development of collateral vessels to revascularise ischemic territories. Alternatively, in cancer treatments, an inhibition of angiogenesis is aimed at.

Administration of angiogenic cytokines was recently proposed as an alternative to surgery or percutaneous transluminal coronary angioplasty (PTCA) for patients suffering from ischemic cardiomyopathy. Several different angiogenic activators have been described sofar. These include, but are not limited to FGF, VEGF and HGF. These growthfactors bind and activate specific receptor tyrosine kinases within the endothelial cells that are coupled to a variety of signal transduction pathways.

Nevertheless, this approach is hampered by two major limitations:

the presence of a diseased and dysfunctional endothelium in ischemic tissue alters its sensitivity to angiogenic cytokines and renders their use difficult to standardise; many contradictory reports on angiogenic properties of these cytokines stemmed from inconsistencies of their effects in vitro versus in vivo, or according to the dose used; and,

the need to maintain a high local concentration of cytokines is a special challenge given numerous side effects, i.e. hypotension, consecutive to the administration of high bolus doses of angiogenic factors.

NO is a well-known relaxing factor and plays a key role in many biological processes. NO is produced in endothelial cells by a Ca²⁺/calmodulin-dependent enzyme, the endothelial isoform nitric oxide synthase (eNOS). eNOS is located in plasmalemmal invaginations termed caveolae, and specifically interacts with cell-specific isoforms of caveolin, the structural protein of caveolae (Feron et al., 1996. J Biol Chem; 271, 22810-22814).

In WO99/22773, the use of metastatic sequences such as caveolin has been suggested in order to treat metastatic diseases. In this approach, the target is to reach the metastatic cells or cells predisposed to metastasis. Decreasing the expression of caveolin was proposed as a possible treatment for cancer.

Some members of the cholesterol/NO pathway as described by Feron et al. seemed to play a role in angiogenesis. For example, Murohara et al. (1998 J. Clin. Inv. 11: 2567-2578) describes that VEGF-mediated angiogenesis is modulated by eNOS. In addition, Kong et al. (1999, Am. Heart Ass. 110: I39), and WO00/67737 suggest to use HMGCoA reductase inhibitors for the treatment of angiogenesis-related diseases. Although Liu et al. (J. Biol. Chem 274: 15781-15785) suggested a role of caveolins in the angiogenetic response, several problems remain unsolved. For example, it is not clear from these documents if and how caveolin may play a key-role in angiogenesis. Caveolin is a multifunctional protein known to influence many pathways or signal transduction cascades according to the cell type or the tissue origin. Caveolin may, for instance, interact with MAPK, Src-family tyrosine kinases, adenylate cyclase and G protein-coupled receptors (Smart et al., 1999). Consequently, it is not obvious to associate the effect of cytokines with caveolin-regulated NO production.

The present invention aims to provide new compounds, compositions and/or methods which may improve the prevention and/or the treatment of various angiogenesis related diseases or pathologies of mammals, including the human, which do not present the drawbacks of the state of the art.

In a first instance, the present invention illustrates that caveolin-1 influences angiogenesis negatively by inhibiting the NO-formation. Secondly, the invention proposes hsp90 and Akt as new targets for modulating angiogenesis. Finally, the invention illustrates the cooperative effect of caveolin-1, hsp90, Akt and known angiogenetic factors such as statins and VEGF on angiogenesis. The present invention unravels the complex pathways in which caveolin-1, hsp90 and Akt are involved, showing that said molecules are good targets in the treatments of angiogensis-related diseases. The invention give proof of the above-mentioned concept enabling the person skilled in the art to influence angiogenesis accordingly.

### SUMMARY OF THE INVENTION

The present invention is related to a pharmacological composition for modulating angiogenesis comprising a therapeutically effective amount of an angiogenesis modulating compound and a pharmaceutically acceptable excipient, whereby said modulating compound is a recombinant caveolin-1, a nucleic acid encoding the partial or total amino acid sequence of caveolin-1, or an analogue thereof or a pharmacologically acceptable derivative thereof; a compound modulating the expression of caveolin-1; an agonist, an antagonist or a competitive inhibitor of caveolin-1; a recombinant hsp90, a nucleic acid encoding the partial or total amino acid sequence of hsp90, an analogue thereof or a pharmacologically acceptable derivative thereof; a compound modulating the expression of hsp90; an agonist, an antagonist or a competitive inhibitor of hsp90; a recombinant Akt, a nucleic acid encoding the partial or total amino acid sequence of Akt, or an analogue thereof or a pharmacologically acceptable derivative thereof; a compound modulating the expression of Akt; an agonist, an antagonist or a competitive inhibitor of Akt.

With modulation is meant, an increase or decrease. Increase in angiogenesis is beneficial in a variety of ischemic cardiovascular diseases. Decrease in angiogenesis would be beneficial in angiogenesis-dependent tumour growth and metastatic diseases.

According to present invention, angiogenesis can be influenced or modulated efficiently through the modulation of intracellular caveolin-1, hsp90 and/or Akt.

The inventors showed not only that that molecules such as statins decrease caveolin-1 abundance resulting in an increased angiogenesis, they additionally proved that the expression of caveolin (example 1) or caveolin-1 scaffolding domains (example 3) abolishes the pro-angiogenetic effect of atorvastatin in vitro. In addition, the invention illustrates that injection of plasmids encoding for caveolin-1 has an effect on the volume of the growing tumor in vivo (example 2). Using inhibitors of hsp90 and by performing over-expression experiments the invention illustrates that hsp90 is an important target for the modulation of angiogenesis (example 3). Immunoprecipitation and phosphorylation studies of present invention pointed towards the role of Akt in angiogenesis (example 4).

### DETAILED ANALYSIS OF THE INVENTION

Present invention relates to a pharmaceutical composition for modulating angiogenesis comprising a therapeutically effective amount of an angiogenesis modulating compound and a pharmaceutically acceptable excipient, wherein said modulating compound is a recombinant caveolin-1, a nucleic acid encoding the partial or total amino acid sequence of caveolin-1, or a pharmacologically acceptable derivative thereof or an analogue thereof.

For example, the pharmaceutical composition for modulating angiogenesis may comprise a therapeutically effective amount of an angiogenesis modulating compound and a pharmaceutically acceptable excipient, wherein said modulating compound is a nucleic acid encoding the partial or total amino acid sequence of caveolin-1, hsp90, Akt or an analogue thereof. The present invention found surprisingly that expression of caveolin-1 in vitro (example 1 of present invention) and in vivo (example 2 of present invention) abolishes angiogenesis. Indeed, as caveolin-1 is a key element in many major pathways, it is not expected that it would exert this effect in vitro and in vivo situations in such a controlled manner. In addition, example 3 of present invention provides evidence that overexpression of hsp90 in endothelial cells (after transfection with hsp90 cDNA) promiotes eNOS activation and a pro-angiogenic phenotype (capillary tube formation in tridimentional Matrigel in vitro). Likewise, heterologous expression of hsp90 in endothelial cells potentiated the pro-angiogenic effect of atorvastatin in the same model.

Alternatively, the pharmaceutical composition for modulating angiogenesis may comprise a therapeutically effective amount of an angiogenesis modulating compound and a pharmaceutically acceptable excipient, wherein said modulating compound is a recombinant caveolin-1, a recombinant hsp90, a recombinant Akt, or a pharmacologically acceptable derivative thereof. Although the addition of vectors encoding interesting proteins such as caveolin-1, hsp90 and/or Akt is a very convenient way of introducing respective proteins in certain target cells, the present invention does not rule out other ways to introduce said compound into a cell. For example proteins, or more stable derivatives thereof, may be taken up by the cell as such. Nevertheless, the uptake may be limited through the size of the molecule that needs to be taken up by the cell. The smaller the molecule is, the more stable and the more efficient it will be taken up. Therefore, present invention suggest that parts of respective molecules can be given. For example, peptides can be loaded into cells (eg. example 3 of present invention).

With a “pharmacologically acceptable derivative” is meant a functional part of the polypeptide or even a chemical molecule mimicking the structural properties thereof. These molecules can be produced externally and added to the cell or produced intracellularly by adding expression vectors carrying respective coding regions and expression signals allowing the production of active polypeptides (see discussion above).

Inhibition of angiogenesis is especially desired in the treatment of solid tumours and their metastases thereby preventing nutrient and oxygen supply of the cancer cells. Present invention shows that angiogenesis is directly linked to the decrease of endogenous caveolin-1. Inventors therefore suggest the use of caveolin-1 sense sequences for the treatment of cancer. In cases increase in angiogenesis is aimed at, present invention proposes to increase the presence or expression of hsp90 and Akt, but decrease the presence or expression of caveolin-1.

According to present invention, a suitable pharmaceutical excipient, carrier or adjuvant is a common pharmaceutical excipient, carrier or adjuvant well known by the person skilled in the art and used to increase or modulate the therapeutical and/or prophylactic effects of the active compounds according to the invention and/or to decrease the possible side effects of said compound(s).

The pharmaceutical composition according to the invention is prepared according to the methods generally applied by pharmacists and may include solid or liquid non-toxic and pharmaceutically acceptable carrier(s) or vehicle(s).

The percentage of active product/pharmaceutically suitable excipient, carrier or adjuvant can vary within very large ranges, only limited by the tolerance and the level of the habit forming effects of the composition to the mammal (including the human). These limits are particularly determined by the frequency of administration.

According to present invention, the pharmaceutical composition may comprise a recombinant expression vector carrying a nucleic acid of present invention as described above. Which expression signals are needed for the efficient expression of respective proteins is known by a person skilled in the art. Expression can be constitutive or controlled in a specific manner.

The present invention also relates to a pharmacological composition which is able to change the concentration of endogenous caveolin-1, Hsp90 or Akt resulting in the modulation of angiogenesis. Said change can result from the modulation of the expression of respective genes coding for the respective proteins; thereby interfering with the promotor, silencer or activator molecules (or sites) influencing the messenger RNA population; or by influencing the translation or stability of the respective messenger RNA. In addition, the activity of the resulting protein can be regulated post-translationally through for example phosphorylation. Therefore, present invention also relates to a pharmaceutical composition for modulating angiogenesis comprising a therapeutically effective amount of an angiogenesis modulating compound and a pharmaceutically acceptable excipient, wherein said modulating compound is a nucleic acid modulating the expression of caveolin-1 thereby interfering with promoter, silencer or activator molecules (or cites) influencing the caveolin-1, hsp90 or Akt mRNA population, respectively. Alternatively, the invention also relates to a pharmaceutical composition for modulating angiogenesis comprising a therapeutically effective amount of an angiogenesis modulating compound and a pharmaceutically acceptable excipient, wherein said modulating compound is influencing the translation or stability of the caveolin-1, hsp90 or Akt mRNA, respectively. Finally, the invention also relates to a pharmaceutical composition for modulating angiogenesis comprising a therapeutically effective amount of an angiogenesis modulating compound and a pharmaceutically acceptable excipient, wherein said modulating compound is influencing the post-translational modification of the caveolin-1, hsp90 or Akt proteins, respectively. Present invention illustrates that Akt by itself regulates the post-translational modification of eNOS regulating thereby angiogenesis (example 4).

According to an embodiment of present invention the pharmacological composition of present invention for the modulation of angiogenesis may be an antisense nucleic acid sequence able to hybridise with a corresponding nucleotide sequence encoding the caveolin-1, hsp90 and/or Akt thereby antagonising the expression of the caveolin-1, hsp90 and/or Akt protein, respectively, in the cell.

In case where the regulation of caveolin-1 is aimed at, said antisense nucleic acid may hybridise with a corresponding sequence encoding the partial or total amino acid sequence of caveolin-1, and is preferably comprised into a recombinant expression vector, under the control of a regulatory sequence that allows its expression (preferably its high expression) in a transfected cell. Preferably, SEQ ID NO 5 is used as antisense sequence. The vectors that can be used for the integration of said genetic sequence are plasmids or viral vectors (such as adenoviruses), which are able to be used for the transfection of endothelial cells in vitro, in vivo or ex-vivo.

Another embodiment of the invention relates to a pharmacological composition for modulating angiogenesis comprising a therapeutically effective amount of an angiogenesis modulating compound and a pharmaceutically acceptable excipient, wherein said modulating compound is a agonist or an antagonist of caveolin-1, hsp90 and/or Akt, thereby influencing the caveolin-1, hsp90 and/or Akt activity, respectively. Alternatively, the invention also embodies pharmacological composition for modulating angiogenesis comprising a therapeutically effective amount of an angiogenesis modulating compound and a pharmaceutically acceptable excipient, wherein said modulating compound is a agonist or an antagonist of caveolin-1, hsp90 and/or Akt, thereby influencing the caveolin-1, hsp90 and/or Akt activity, respectively. Said antagonists can antagonise the function of the molecule per se or act as a competitive inhibitor by inhibiting the binding of binding partners. A competitive inhibitor can also be defined as being a scavenging molecule or a molecule able to trap one of the caveolin-1, hsp90 and/or Akt molecules. In example 3 of present invention, geldanamycin, an inhibitor of the hsp90 function is used. This example illustrates the use of this inhibitor in inhibiting angiogenesis.

Another possible embodiment of the invention relates to a pharmacological composition for modulating angiogenesis comprising a therapeutically effective amount of an angiogenesis modulating compound and a pharmaceutically acceptable excipient, wherein said modulating compound is a competitive inhibitor of caveolin-1, hsp90 and/or Akt; whereby said inhibitor is a scavenging or trapping molecule.

In cases where the modulation of the caveolin-1 activity is aimed at, said trapping molecule is able to trap the endogenous caveolin-1 preventing its binding to the endothelial isoform nitric oxide synthase (eNOS). Possible compounds according to the invention are short peptidic sequences corresponding to the active sites upon eNOS to caveolin-1 such as described below. Said peptides can be made synthetically or produced in a cellular system. Systems that can be used to produce such proteins are known by the person skilled in the art. According to present invention the pharmaceutical composition for modulating angiogenesis may also comprise a nucleic acid preferably comprised into a vector encoding the partial or total amino acid sequence of eNOS as described above or the eNOS sequence mutated or deleted in the active (caveolin) binding site or an analogue thereof which can increase the concentration of unbound (activated) eNOS. Possible compounds according to the invention are short peptidic sequences corresponding to the active sites (having preferably at least the common pattern SEQ ID NO 4, more preferably the pattern SEQ ID NO 6 to 86) upon eNOS to the caveolin-1 scaffolding domains A and B (described in the following sequences SEQ ID NO 2 and SEQ ID NO 3).

The present invention also relates to a pharmacological composition for modulating angiogenesis comprising a therapeutically effective amount of an angiogenesis modulating compound and a pharmaceutically acceptable excipient, wherein said modulating compound is a trapping molecule able to trap the endogenous eNOS. Said molecule mimicks caveolin-1, blocking its active site and preventing the NO synthesis. Possible compounds according to the invention are short peptidic sequences corresponding to the active sites upon caveolin-1 to the eNOS such as described below.

According to present invention, the pharmacological composition for modulating angiogenesis may be a nucleotide sequence encoding the partial or total amino acid sequence of caveolin-1. Alternatively, an analogue to these can be used. Increase in caveolin-1 concentration in the cell results in the increase scavenging of the endogenous eNOS. Scavenging eNOS decreases the production of NO thereby carrying an inhibiting effect on angiogenesis.

As an example of the invention, said partial amino acid sequence may comprise the caveolin-1 scaffolding domains A and/or B of caveolin-1 (described in the following sequences SEQ ID NO 2 and SEQ ID NO 3) or portions thereof able to bind selectively upon the endothelial isoform of nitric oxide synthase (eNOS).

As caveolin-1 is a protein/peptide (nucleotide sequence of caveolin-1 enclosed see SEQ ID NO 1), said analogs or compounds can be derivatives of said prdtein/peptide, peptidomimetics or homologous peptides that comprise the deletion or the replacement of one or more amino acids in the original caveolin-1 sequence, or antibodies directed against the ligand binding site epitopes of the active site(s) upon the endothelial isoform of nitric oxide synthase, or anti-idiotypic antibodies directed against particular antibodies directed against the specific portions of caveolin-1 (scaffolding domain A and/or B of caveolin-1 (SEQ ID NO 2 and SEQ ID NO 3) binding the active site(s) (of caveolin-1) upon the endothelial isoform of nitric oxide synthase. Said antibodies can be raised to caveolin-1 fragments and analogs both in the unnatural occurring form or in the unrecombined form. Similar approaches can be followed for the hsp90 and Akt proteins. The present inventors found surprisingly that peptides with sequences comprising the caveolin scaffolding domain (CSD)-sequences that mimmick endogenous caveolin binding to (and inhibition of) eNOS- can abolish the pro-angiogenic effect of atorvastatin on cultured endothelial cells (example 3, FIG. 9); likewise, CSD peptides decrease the ability of atorvastatin to activate the recruitment, on the eNOS multi-protein complex, of hsp90 and P-Akt (the phosphorylated, active form of the protein kinase Akt, an activator of eNOS), both required to activate eNOS and promote the pro-angiogenic phenotype in endothelial cells (example 3, FIG. 8).

In particular the present invention also relates to a composition comprising a statin and/or VEGF and hsp90 and/or Akt, or a pharmacologically acceptable derivative thereof as a combined preparation for simultaneous, separate or sequential use in a therapy in an angiogenesis-related disease. In addition, the present invention embodies the use of hsp90 and/or Akt, or a pharmacologically acceptable derivative thereof, for the modulation of proangiogenic actions of statins and/or VEGF. In both statements, said composition, hsp90 and Akt refer to respective recombinant proteins; to respective nucleic acids coding for respective proteins; and; to agonist, antagonists or competitive inhibitors of respective proteins. Present invention gives a clear view on the fact that there is an additional synergistic effect of said compounds on the already known effects of statins and VEGF on angiogenesis (examples 3 and 4 of present invention).

The present invention also provides a method of modulating angiogenesis comprising the step of trapping endogenous caveolin-1 by a trapping compound thereby preventing its binding to the endothelial isoform nitric oxide synthase (eNOS).

For example, according to present invention, said trapping compound may comprise an amino acid sequence pattern as described in SEQ ID NO 4, preferably comprising the amino acid sequence pattern as described in SEQ ID NO 6 to SEQ ID NO 86.

Alternatively, the present invention also relates to a method of modulating angiogenesis comprising the step of trapping endogenous eNOS by a trapping compound whereby said trapping compound is mimicks caveolin-1.

For example, according to present invention, the latter trapping compounds may comprise an amino acid sequence pattern as described in SEQ ID NO 2 or SEQ ID NO 3.

Present invention also relates to a method of modulating angiogenesis comprising the step of overexpressing or improving the activity of caveolin-1, hsp90 and/or Akt. Alternatively, present invention relates to a method of modulating angiogenesis comprising the step of reducing the abundance and/or activity of caveolin-1, hsp90 and/or Akt.

The present invention also provides a method for the treatment of angiogenesis related diseases comprising the step of administering a therapeutically effective amount of an angiogenesis modulating compound to an individual in need thereof, wherein said modulating compound is a recombinant caveolin-1, a nucleic acid encoding the partial or total amino acid sequence of caveolin-1, or an analogue thereof or a pharmacologically acceptable derivative thereof; a compound modulating the expression of caveolin-1; an agonist, an antagonist or a competitive inhibitor of caveolin-1; a recombinant hsp90, a nucleic acid encoding the partial or total amino acid sequence of hsp90 or an analogue thereof or a pharmacologically acceptable derivative thereof; a compound modulating the expression of hsp90; an agonist, an antagonist or a competitive inhibitor of hsp90; a recombinant Akt, a nucleic acid encoding the partial or total amino acid sequence of Akt, or an analogue thereof or a pharmacologically acceptable derivative thereof; a compound modulating the expression of Akt; an agonist, an antagonist or a competitive inhibitor of Akt.

The present invention relates to a method according to present invention for the treatment of angiogenesis related diseases such as angiogenesis-dependent tumour growth and metastatic diseases, ischemic heart and peripheral vascular diseases including cerebral diseases and wound healing. The decrease or the increase of nitric oxide results in a modification of neo-angiogenesis which has prophylactic or therapeutic properties in specific pathologies and diseases of mammals (including the human), and preferably selected from the group consisting of: high blood pressure, cardiac insufficiency, cardiac decompensation, ischemic cardiomyopathy, dilated or post-transplantation cardiomyopathies, angina pectoris (including instable angina), coronary spasm, post-transplantation coronaropathy, hypercholesterolemia, hyperlipidemia, hypertriglyceridemia, vascular side effects of diabetes melitus (insulino-dependent or not), vascular side effects of chronic renal insufficiency (uremia), endothelial dysfunction of various origins (atherosclerosis, smoke-addiction, syndrome X, obesity, hypertension, dyslipidemia, resistance to insulin), systemic or auto-immune vasculitis, hyperhomocysteinemia, buerger angeitis, post-angioplasty coronary restenosis, peripheral vascular disease, thrombo-embolic disease, deep or superficial vein thrombosis, atherosclerosis with arterial insufficiency in a vascular area (ischemia, including celebral ischemia, coronary ischemia, . . . ), pulmonary arterial hypertension, side effects of hemodialysis or peritoneal dialysis, various neoplastic diseases (including carcinogenesis, tumoural development and metastases proliferation), resistance of malignant neoplastic tumours to radio or chemotherapy, bladder cancer metastatic or not (cystadenocarcinoma), angiosarcoma, proliferative retinopathies, wound healing or a mixing thereof. For cancer treatment inhibition of this angiogenesis is required and therefore increase of caveolin-1 is pursued.

In particular, the present invention also provides a diagnostic kit for the testing of a compound or a composition for their ability to modulate angiogenesis comprising an angiogenesis modulating compound wherein said modulating compound is a recombinant caveolin-1, a nucleic acid encoding the partial or total amino acid sequence of caveolin-1, or an analogue thereof or a pharmacologically acceptable derivative thereof; a compound modulating the expression of caveolin-1; an agonist, an antagonist or a competitive inhibitor of caveolin-1; a recombinant hsp90, a nucleic acid encoding the partial or total amino acid sequence of hsp90, or an analogue thereof or a pharmacologically acceptable derivative thereof; a compound modulating the expression of hsp90; an agonist, an antagonist or a competitive inhibitor of hsp90; a recombinant Akt, a nucleic acid encoding the partial or total amino acid sequence of Akt, or an analogue thereof or a pharmacologically acceptable derivative thereof; a compound modulating the expression of Akt; an agonist, an antagonist or a competitive inhibitor of Akt.

The following examples and figures merely serve to illustrate the invention and are by no way to be understood as limiting the present invention

### LEGEND OF THE FIGURES

FIG. 1: A. Pavimentous organization of endothelial cells cultured on dishes. B. Tube formation in the <<3D>> Matrigel model. C. Tube formation in the <<sandwich>> Matrigel model. D. Inhibition of tube formation in caveolin-overexpressing endothelial cells in the <<sandwich>> model.

FIG. 2: The use of a plasmid encoding for caveolin leads to a significant decrease in tumor volume when injected either intra-venously (IV) or intra-tumoral (IT).

FIG. 3: Immunoblots performed on total lysates obtained from the TLT tumors dissected from mice at day 6 after transfection (see Figure A). The phosphorylation of eNOS on serine 1177 (used as a hallmark of eNOS activation) was inhibited in mice injected with the caveolin plasmid. Of note, eNOS is an endothelial enzyme which is not expressed in tumor cells and these immunoblot data therefore indicate that the effect of caveolin transfection targets the endothelium of the tumor vessels.

FIG. 4: Atorvastatin stimulates NO-dependent angiogenesis in CMECs and AoECs. Shown are representative pictures of freshly isolated CMECs plated on Matrigel for 3 hours (A through C) and of AoECs grown in Matrigel for 5 days and placed under serum-free conditions for 2 more days (D through F). The following treatments were performed as detailed in Results: A and D, vehicle; B and E, atorvastatin (1 _mol/L); C and F, atorvastatin_L-NAME (5 mmol/L). Inset in panel D, Typical endothelial tube organization arising from a cultured aortic ring.

FIG. 5: Atorvastatin decreases the extent of the caveolin/eNOS interaction in AoECs but not in CMECs. ECs were recovered from Matrigel after exposure to atorvastatin (St) (1 μmol/L) or vehicle (V). A, CMEC and AoEC lysates were immunoprecipitated with eNOS (left) or caveolin (right) antibodies and analyzed by IB with caveolin (top) and eNOS (bottom) antibodies. B, Lysates were also directly analyzed by IB with caveolin antibodies. Lysates from outgrowing CoMECs and isolated AECs were also used for comparison. These experiments were repeated 2 to 3 times with similar results. C, Densitometric analysis of caveolin IB presented in panel B; expression levels are normalized to number of cells (open bars, control; hatched bars, statin). D, Effects of atorvastatin treatment on cholesterol efflux (ie, serum-stimulated decrease in [³H]cholesterol cell content) from AECs and CMECs. *P<0.01 (n=3) vs corresponding control conditions.

FIG. 6: Atorvatatin promotes the hsp90/eNOS interaction and the Ser1177 eNOS phosphorylation. ECs were recovered from Matrigel after exposure to vehicle (V) or atorvastatin (St) (1 μmol/L), in the presence or absence of geldanamycin (1 μg/mL) or the PI(3)K inhibitor LY294002 (10 μmol/L). Lysates were immunoprecipitated with eNOS antibodies and immunoblotted with hsp90 (top) and eNOS (middle) antibodies. Lysates were also directly immunoblotted with antibodies directed against phospho-Ser1177-eNOS (bottom). These experiments were repeated twice with similar results.

FIG. 7: Hsp90 is a critical regulator of the Akt and eNOS phosphorylations in response to atorvastatin. HUVECs were cultured to confluence, placed in serum-free medium for 12 hours, and then exposed for 2 hours (or the indicated period of time) to atorvastatin (1 μmol/L) before lysis. Statin treatment was preceded or not by addition of the PI(3)K inhibitor LY294002 (10 μmol/L), the hsp90 inhibitor geldanamycin (1 μg/mL), and the phosphatase inhibitors okadaic acid (100 nmol/L) and calyculin (10 nmol/L). A, Cell lysates were immunoblotted with antibodies directed against phospho-Ser1177-eNOS and phospho-Ser473-Akt (top). Lysates were also immunoprecipitated with eNOS antibodies and analyzed by IB with phospho-Ser473-Akt and eNOS antibodies (bottom). Note that quantitative analysis of some of these assays is presented in FIG. 6B. B, Time course of Akt and eNOS phosphorylations (bottom) and of Akt/hsp90, eNOS/Akt, and eNOS/hsp90 interactions (top), as revealed by direct IB or after IP, respectively. C, Effects of statin on hsp90 tyrosine phosphorylation; shown are time course (top) and dose dependency (bottom). Hsp90 immunoprecipitates were analyzed by IB with anti-phosphotyrosine and hsp90 antibodies. These experiments were repeated 2 to 3 times with similar results.

FIG. 8: Dissection of statin effects leading to hsp90-dependent phosphorylation of eNOS by Akt in HUVECs. HUVECs were cultured and incubated with atorvastatin (at time t=0) as detailed in FIG. 4 legend. In some experiments, HUVECs were either transfected (t-48 hours) to express recombinant hsp90 or reversibly permeabilized (t-3 hours) to introduce large amounts of CSD peptide, as detailed in Materials and Methods. Cells were also preincubated (t-60 minutes) with the HMGCoA reductase downstream product mevalonate (1 mmol/L) or the calcium chelator BAPTA-AM (20 μmol/L). Corresponding cell lysates were immunoblotted with antibodies directed against hsp90, phospho-Ser1177-eNOS, and phospho-Ser473-Akt (top). Protein-protein interactions were also examined by co-IP as follows: lysates were immunoprecipitated with eNOS antibodies and analyzed by IB with hsp90, phospho-Ser473-Akt, and eNOS antibodies (bottom); in the vehicle condition (V), a faint signal for hsp90 was detected in the eNOS IP only on longer film exposures. These experiments were repeated 2 to 3 times with similar results. Note that quantitative analysis of some of these assays is presented in FIG. 6B.

FIG. 9: Hsp90 and caveolin oppositely modulate angiogenesis in HUVECs. HUVECs were cultured as detailed in FIG. 4 legend and then distributed in 24-well multidishes containing Matrigel, as detailed in Materials and Methods. In some experiments, before the angiogenesis assay, HUVECs were either transfected with hsp90-encoding plasmid or loaded with CSD peptide, as detailed in FIG. 5 legend. A, Representative pictures of HUVECs plated on Matrigel for 2, 5, and 24 hours in the absence or presence of 0.1 μmol/L atorvastatin (added at time t=0). B, Quantification of statin- and/or hsp90-induced tube formation combined to densitometric analysis of eNOS phosphorylation and eNOS/phospho-Akt interaction under corresponding conditions (from FIGS. 4 and 5). *P<0.01 vs corresponding parameters under statin (St) conditions.

FIG. 10: Time course of VEGF-induced caveolin/eNOS dissociation, eNOS/hsp90 association, and phosphorylation of Akt and eNOS in EC. HUVECs were exposed to VEGF for the indicated periods of time. A, in some experiments, lysates were immunoprecipitated with caveolin or eNOS antibodies. Caveolin immunoprecipitation was analyzed by immunoblotting with eNOS antibodies (top) and eNOS immunoprecipitation by immunoblotting with hsp90 antibodies (bottom). B, lysates were also directly immunoblotted with antibodies directed against phospho-Ser¹¹⁷⁷-eNOS (top) or phospho-Ser⁴⁷³-Akt (bottom). Note that the PI3K inhibitor LY294002 blocked both eNOS and Akt phosphorylation upon 30 min VEGF stimulation (right panels) C, densitometric analysis of three independent experiments (as shown in B) demonstrates the lag between the time courses of VEGF-induced Akt and eNOS phosphorylation (*, p<0.01 versus phospho-Akt time control).

FIG. 11: Recruitment of Akt in the hsp90-eNOS complex pro-motes eNOS phosphorylation in EC exposed to VEGF and in insulin-stimulated cardiac myocytes. A, HUVECs were exposed to VEGF for the indicated periods of time, and then rapidly collected. Corresponding lysates were immunoprecipitation with eNOS antibodies, resolved by SDS-polyacrylamide gel electrophoresis and analyzed by immunoblotting with Akt antibodies. Akt was detectable in the immunoprecipitation as a function of time exposure in cells treated with VEGF (top) but not in cells exposed to VEGF and geldanamycin (bottom). B, lysates from 5-min VEGF-treated EC (preincubated or not with geldanamycin) were submitted to the reverse immunoprecipitation with either hsp90 or Akt antibodies, and analyzed by immunoblotting for eNOS expression. The supernatants (Spnt) of these immunoprecipitations were submitted to another immunoprecipitation with eNOS antibodies before immunoblotting analysis to estimate their residual eNOS content; the proportion of detected eNOS (versus total eNOS pool) is indicated for each couple of lanes. C, lysates from VEGF/geldanamycin-treated EC were also directly analyzed by immunoblotting with antibodies directed against phospho-Ser¹¹⁷⁷-eNOS (top) or phospho-Ser⁴⁷³-Akt (bottom). As a positive control for phosphorylation, lysates from EC exposed for 30 min to VEGF in the absence of geldanamycin were analyzed in parallel (see sixth lane). D, the Akt activity was measured as the extent of phospho-GSK-3 detected by immunoblotting after incubation of a GSK-3 fusion protein and Akt immunoprecipitates from the lysates of EC exposed for 30 min to VEGF in the absence (veh.) or presence of geldanamycin (gelda) or LY294002 (LY) versus unstimulated control cells (CTL). E, neonatal mouse cardiac myocytes were exposed to insulin for 5 min in the absence (veh.) or presence of geldanamycin (gelda). Corresponding lysates as well as lysates from control, unstimulated cells (CTL) were analyzed by immunoblotting with antibodies directed against phospho-Akt, phospho-eNOS, and phospho-GSK-3. These experiments were repeated two to four times with similar results.

FIG. 12: Evidence that Ser¹¹⁷⁷ functionally mediates the hsp90- and Akt-dependent increase in NO production in transfected COS cells. COS cells were co-transfected with the constructs encoding for wild-type eNOS (black bars) or S1177A eNOS mutant (open bars) and plasmids for Myc-tagged active Akt (T308D/S473D), and/or hsp90, or the corresponding vector. NO₂⁻ accumulation in the extracellular medium (collected in the 44-48-h interval after transfection) was determined by electrochemical detection. Data are expressed as percentages (+/−S.E.) of the NO₂⁻ production in cells expressing WT-eNOS alone (top); *, p<0.01, n=3. The expression of wild-type and mutant eNOS, active Akt, and hsp90 was controlled by immunoblotting using anti-eNOS, anti-Myc tag, and anti-hsp90 antibodies, respectively (bottom).

FIG. 13: Akt recruitment and phosphorylation of eNOS require the initial increase in [Ca²⁺]i induced by VEGF. HUVECs were exposed to VEGF in the absence or presence of the calcium chelator BAPTA. A, EC exposure to VEGF led to an increase in [Ca²⁺]i that is completely prevented in the presence of BAPTA. Data are expressed as the Fura-2 fluorescence signal ratio which is a reflection of changes in [Ca²⁺]i; at the concentration used (20 μM BAPTA-AM), BAPTA pre-vented the VEGF-induced calcium transient but had no effect on the basal level of [Ca²⁺]i. These experiments were repeated 3-4 times with similar results. B, BAPTA-exposed EC were also collected at the indicated periods of time for further analyses. Caveolin immunoprecipitation were analyzed by immunoblotting with eNOS antibodies (top) and eNOS immunoprecipitation by immunoblotting with Akt antibodies (middle). Lysates were also directly immunoblotted with antibodies directed against phospho-Ser 1177-eNOS (bottom). For each experiment described in B, lysates from EC exposed for 30 min to VEGF in the absence of BAPTA were also analyzed in parallel as controls (see sixth lane). These experiment were repeated twice with similar results.

FIG. 14: The early VEGF-induced increase in [Ca²⁺]i is required for the long-term phosphorylation-dependent eNOS activation. Levels of NO2_accumulated in medium bathing EC for the indicated time intervals following VEGF stimulation were determined by electro-chemical detection. A, bar graph illustrates the absolute amounts of NO₂⁻, expressed as mean +/−S.E., accumulated in 5-min intervals. In the first bar (0-5 min interval), the proportion of NO₂⁻ produced in the first 2 min is indicated in black; note also that for the 55-60-min interval, the amounts of NO₂⁻ were below the detection level (b.d.l.). B, bar graph illustrates the relative rates of NO₂⁻ production per min: data areexpressed in % (+/−S.E.) of NO₂₋ produced (per min) in the first 2 min of VEGF stimulation in the absence of any additional treatment (first open bar). Note that the absolute amounts of NO₂⁻ produced in the early (open bars) and late (black bars) phases can be obtained by multiplying the depicted relative rates by a factor 2 and 25, respectively (in control conditions, the absolute amount of NO₂⁻ produced in the 5-30-min period amounted to 5.5-fold the amount of NO₂⁻ derived from the first 2-min VEGF exposure). Shown are the effects of geldanamycin, LY294002, and the Ca²⁺ chelator BAPTA on the early and late VEGF-stimulated NO production. The effects of a transient increase in [Ca²⁺]I resulting from a short exposure to the calcium ionophore A23187 are also presented for comparison (note the very low level of NO production in the 5-30-min period versus VEGF stimulation); *, p<0.01 versus corresponding time control, n=3. C, in some conditions of the assay described in A, EC were collected and lysates analyzed for the extent of CaM associated to eNOS and the level of Akt phosphorylation. Accordingly, EC exposed (or not) to VEGF (in the presence or in the absence of BAPTA) or to a transient pulse of A23187 (see “Results”) were collected after 2 or 30 min and the corresponding lysates were analyzed. Top, eNOS immunoprecipitation were analyzed by immunoblotting with CaM antibodies. Bottom, lysates were also directly immunoblotting with antibodies directed against phospho-Akt. These experiments were repeated twice with similar results.

The invention is further elucidated in the examples hereunder:

### EXAMPLE 1

### Illustrates the Anti-Angiogenic Effect Observed In Caveolin-Overexpressing Cells In Vitro.

The inventors have developed different techniques to evaluate angiogenesis in vitro and used some of them to test the possibility to modulate NO-dependent angiogenesis by altering caveolin abundance.

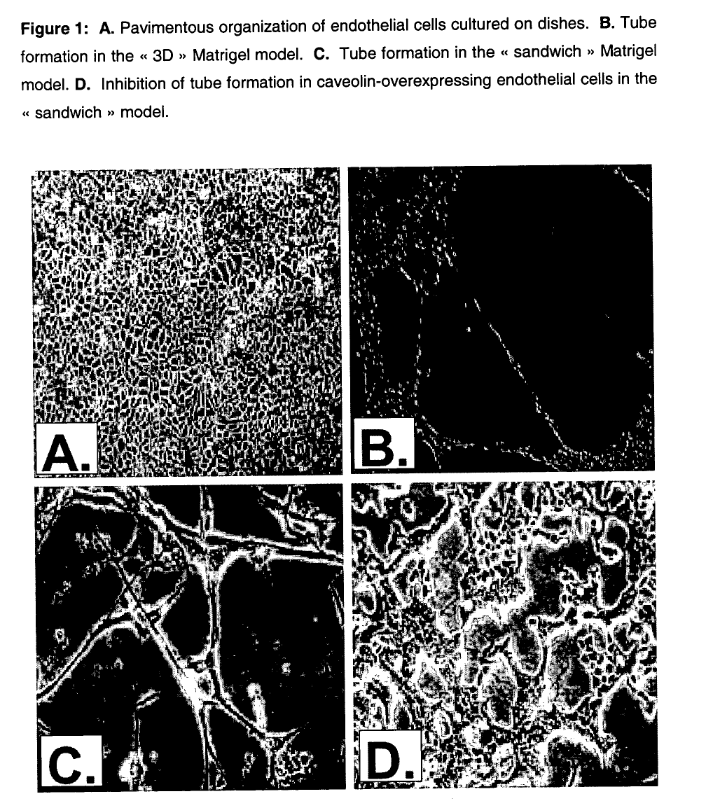

Indeed, while endothelial cells culture leads to pavimentous organization when plated on dishes (FIG. 1A), endothelial cells progressively form tubes when cultured within gels of collagen, fibrin or Matrigel®. Accordingly, in the “3-D model”, endothelial cells are mixed to the matrix before gelification and tube-like structures are obtained after 48-72 hours (FIG. 1B); this model, however, reduces the efficiency of transfection. In the “sandwich model”, cells are first cultured to confluence on a first layer of matrix, and are then covered by a second matrix layer; cells are easily transfected in the interval preceding the addition of the upper layer. The latter technique allows the formation of endothelial tubes in 3-6 hours; the tube structures are mature 12 hours after induction (FIG. 1C) and then start to regress.

When endothelial cells were first transfected with a caveolin-encoding vector (24 hours before addition of the second matrix layer), the formation of tube structures was dramatically inhibited (FIG. 1D). Importantly, SNAP, a NO donor agent, was shown to correct this anti-angiogenic effect observed in caveolin-overexpressing cells. Yet, in non-transfected cells, both SNAP and the HMGCoA reductase inhibitor atorvastatin accelerated the endothelial tube formation in this model of in vitro angiogenesis. Also, in the same model, NOS inhibitors were shown to mimick the effect of caveolin overexpression, i.e. by slowing down the tube formation. Of note, when endothelial cells were transfected with an irrelevant construct (β-galactosidase-encoding), no change in the rate and extent of tube formation was observed.

### EXAMPLE 2

### Shows That Caveolin Exerts An Inhibitory Activity On Angiogenesis In An In Vivo Model of Angiogenesis-Dependent Tumor Growth.

Hepatocarcinoma cells are able to grow as a tumor after injection in the leg of mice is dependent on the ability of endothelial cells to develop new vessels to “feed” the tumor. This vessel development depends on the capacity of endothelial cells to produce NO through eNOS activation in these cells, itself regulated by the post-translational modulators it was previously identified in the in vitro experiments, e.g. caveolin-1.

Tumor mouse model: 30-40 g male NMRI mice received an IP injection of TLT cells (hepatocarcioma). Ascite cells were recovered in physiological serum and IM-injected in the posterior left leg of ketamine/xylazin anesthetized mice. Tumors grew for 9-12 days to reach 8±1 mm of diameter, the original diameter for treatment (caveolin plasmid injection) at day 0.

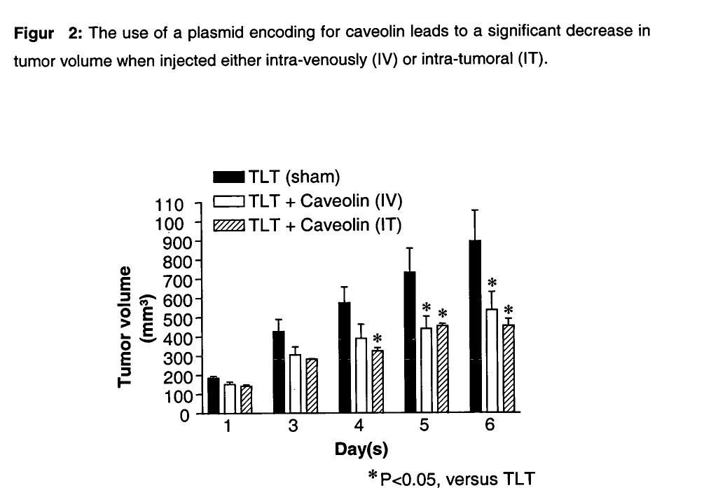

The effect of injection of plasmids encoding caveolin-1 has been evaluated on the volume of the growing tumor after tumor implantation in the leg (FIG. 2). Caveolin-encoding plasmids were injected either intravenously or directly in the tumor. The working hypothesis was that heterologous over-expression of caveolin-1 in endothelial cells will inhibit eNOS activation in endothelial cells, with a subsequent inhibition of angiogenesis that will result in a decrease in tumor growth.

After 5 days, a significantly reduction of the tumor was detected when the caveolin-encoding plasmid was injected both intra-venously or intra-tumoral. This illustrates that caveolin expression indeed results in the decrease of the tumors in vivo. Accordingly, the use of recombinant caveolin-1 may result in a similar result.

Since the recombinant caveolin-1 was <<tagged>> with hemagglutinin antigen (HA), the amount of recombinant caveolin-1 in the tumor endothelial cells was immunologically detected and quantitated. Therefore, it was established that the extent of recombinant (HA-tagged-) caveolin expression was inversely correlated to the tumor volume.

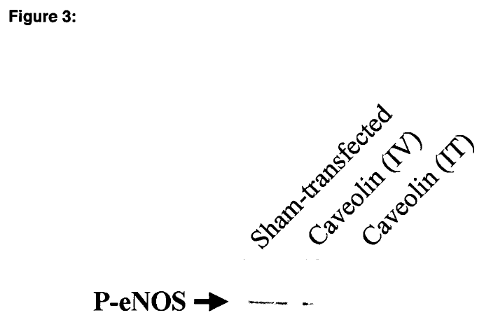

To assess the cellular specificity of the performed caveolin-1 transfection approach, the impact of heterologous cav-1 expression on eNOS phosphorylation was evaluated, an index of eNOS activation. Since eNOS is exclusively expressed in endothelial cells, any change in its phosphorylation (activation) is indicative of an effect of caveolin-1 in the endothelial cells of the tumors (FIG. 3).

FIG. 3 shows that the phosphorylation of eNOS was inhibited in mice when injected with the caveolin-1 encoding plasmid.

This example demonstrates that injection of caveolin-encoding plasmid leads to its expression in tumor endothelial cells and consecutively, prevents the pro-angiogenic activation of eNOS, thereby dramatically slowing down tumor development.

### EXAMPLE 3

### Demonstrates That Hsp90 and Caveolin Are Key Targets for the Proangiogenic Nitric Oxide-Mediated Effects of Statins.

3-Hydroxy-3-methylglutaryl (HMG)-coenzyme A reductase inhibitors or statins exert direct beneficial effects on the endothelium in part through an increase in nitric oxide (NO) production. Posttranslational modifications of the endothelial NO synthase (eNOS) were analysed and tested if they could account for the proangiogenic effects of statins. Endothelial cells (ECs) were isolated from cardiac microvasculature, aorta, and umbilical veins, as well as dissected microvessels and aortic rings, that were cultured on reconstituted basement membrane matrix (Matrigel). Tube or precapillary formation was evaluated after statin treatment, in parallel with immunoblotting and immunoprecipitation experiments. Atorvastatin stimulated NO-dependent angiogenesis from both isolated and outgrowing (vessel-derived) ECs, independently of changes in eNOS expression. In macro- but not microvascular ECs, atorvastatin stabilized tube formation through a decrease in caveolin abundance and its inhibitory interaction with eNOS. The chaperone protein hsp90 was identified as a key target for the proangiogenic effects of statins. By using geldanamycin, an inhibitor of hsp90 function, and overexpression of recombinant hsp90, it was documented that the statin-induced phosphorylation of eNOS on Ser1177 was directly dependent on the ability of hsp90 to recruit Akt in the eNOS complex. Finally, it was shown that statin promoted the tyrosine phosphorylation of hsp90 and the direct interaction of hsp90 with Akt, which further potentiated the NO-dependent angiogenic processes. This example provides new mechanistic insights into the NO-mediated angiogenic effects of statins and underscores the potential of these drugs and other modulators of hsp90 and caveolin abundance to promote neovascularization in disease states associated or not with atherosclerosis. The present invention provides new mechanistic insights into the NO-mediated angiogenic effects of statins and opens new perspectives in the development of therapeutic strategies targeting cardiac or peripheral angiogenesis.

The cell culture was performed as follows: Mouse cardiac microvascular ECs (CMECs) and human umbilical vein ECs (HUVECs) were freshly prepared according to standard isolation procedures. Bovine aortic ECs (AECs) were purchased from Clonetics and used at early passages. Serum-starved ECs were usually exposed to atorvastatin (0.1 to 1 μmol/L) after a 30- to 60-minute preincubation with various pharmacological modulators, as follows (from Sigma except when indicated): 5 mmol/L N^{G}-nitro-L-arginine methyl ester (L-NAME), 1 mmol/L mevalonate, 20 μmol/L BAPTA-AM, 1 μg/mL geldanamycin (Invitrogen Life Technologies), 10 μmol/L LY294002, 100 nmol/L okadaic acid, and 10 nmol/L calyculin.

Aorta-derived ECs (AoECs) and coronary microvessel-derived ECs (CoMECs) were obtained by incubating small rat aorta rings (1 mm length) and human microvessels (dissected from endocardial biopsies), respectively, for 5 to 7 days in Matrigel (see below) in the presence of 20% FCS/DMEM. In this model, atorvastatin was added from day 0 to day 5, and the medium was replaced every 24 hours with fresh drug solution. Cultures of AoECs and CoMECs organized in a network were then challenged at day 5 by incubation under serum-free conditions for 24 to 48 hours with or without some of the drugs mentioned above.

The identity and purity of the different ECs used in this example were routinely validated by indirect immunofluorescence using von Willebrand factor antibodies. In addition, serum-stimulated cholesterol efflux measurements were carried out to evaluate the functional pool of caveolin in the different ECs (see online Materials and Methods available at http://www.circresaha.org).

The Angiogenesis Assay on Matrigel was performed as follows: The formation of capillary-like structures was assessed by plating (CMECs, AECs, and HUVECs) on or culturing (AoECs, CoMECs) in basement membrane matrix preparation (growth factor-reduced Matrigel [Becton Dickinson]) distributed in 24-well multidishes. Tube formation was observed using an inverted phase-contrast microscope (Zeiss Axiovert 25), and images were captured with a videographic system (Pixera Pro). The tube formation index was determined by measuring the length of tubes in random fields from each well using the Image J program. Statistical analyses were made using the Student t test or one-way ANOVA where appropriate.

In some experiments, ECs were recovered from Matrigel after a 5-minute incubation with 50 mg/mL collagenase under constant agitation, followed by two sequences of washing/centrifugation (2000 rpm for 5 minutes at 4° C.).

Transfection and peptide loading was performed by reversible permeabilization: HUVECs were transfected with the hsp90 cDNA (a gift from Dr W. C. Sessa, Yale University, New Haven, Conn.) using Lipofectamine (Invitrogen) according to the manufacturer's protocol; an irrelevant plasmid encoding β-galactosidase was used as a control to obtain identical amounts of transfected DNA in each condition. HUVECs were also reversibly permeabilized, as previously reported (Feron et al. J Biol Chem. 1998; 273:30249-30254), to introduce synthetic peptides derived from the caveolin scaffolding domain (CSD).

Immunoprecipitation (IP) and Immunoblotting (IB) was performed as follows: ECs were processed for IB or IP as described previously (Feron et al. Circulation. 2001;103:113-118, Brouet et al. J Biol Chem. 2001; 276:32663-32669); antibodies were from BD Transduction Labs, except phospho-eNOS antibody, which was from New England Biolabs.

An expanded Materials and Methods section can be found in an online data supplement available at http://www.circresaha.org.

Hereunder is proven that atorvastatin stimulates NO-dependent angiogenesis in two models of EC primary cultures.

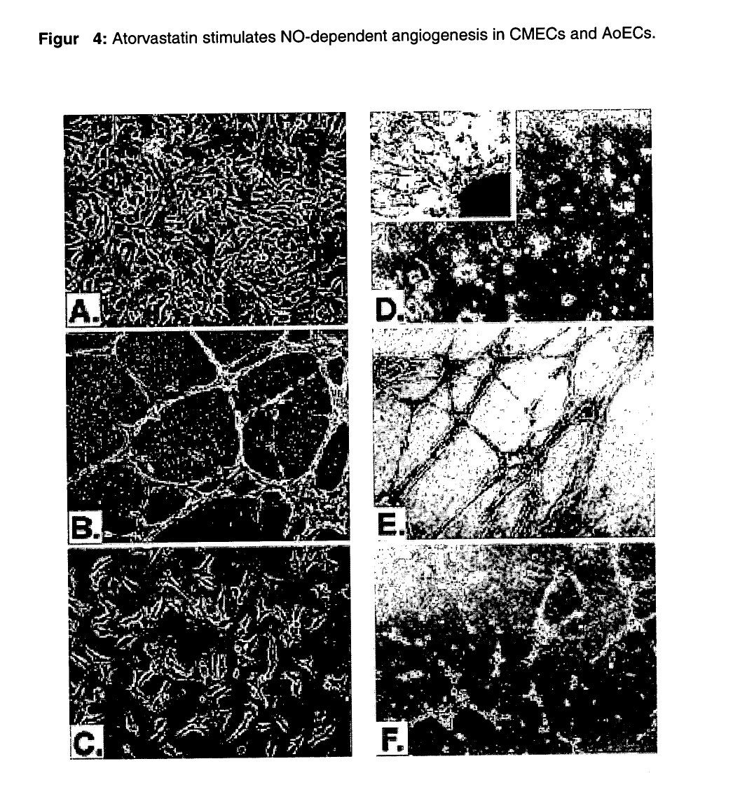

The ability of statins to stimulate and/or stabilize tube formation from CMECs and AoECs cultured on Matrigel was studied. As shown in FIG. 4B, exposure of freshly isolated CMECs to atorvastatin rapidly led to the formation of capillary-like structures on the Matrigel surface; this proangiogenic effect was maximal after 3 hours and almost completely blocked by the coincubation with the NO synthase inhibitor L-NAME (FIG. 4C). Atorvastatin similarly promoted AoEC network organization but with a very distinct time course because of the nature of this model of tissue culture. Aortic rings had, indeed, to be cultured for 5 to 7 days in Matrigel to exhibit EC proliferation and tube formation (see FIG. 4D, inset). The presence of atorvastatin during this period of cell growth did not significantly influence serum-driven formation of the AoEC network. However, when the vessel culture was deprived of serum for 48 hours, pretreatment with statins dramatically stabilized the network organization (FIG. 4E), whereas the precapillaries formed in the absence of statins completely degenerated (FIG. 4D). Importantly, the addition of L-NAME at the time of serum deprivation abrogated the beneficial effects of statin pretreatment (FIG. 4F).

Hereunder is illustrated that atorvastatin decreases the extent of the caveolin/eNOS interaction in AoECs but not in CMECs.

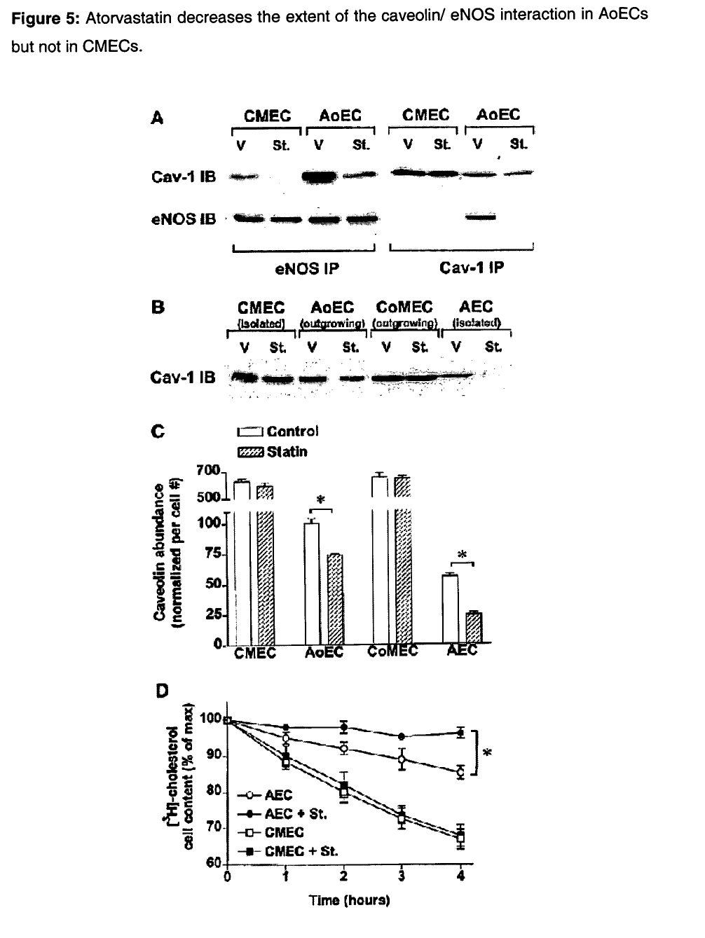

Because atorvastatin treatment did not alter the expression of eNOS in either CMECs or AoECs (see FIG. 5A, bottom left), it was examined whether the NO-mediated angiogenic process reported in FIG. 4 could, instead, originate from the modulation by statins of posttranslational modifications of eNOS. eNOS activity has, indeed, been shown to be either decreased by the interaction of the enzyme with the scaffolding protein caveolin or increased after recruitment of the heat shock protein, hsp90. Accordingly, ECs were recovered from Matrigel after exposure to atorvastatin (St) or vehicle (V), and the extent of caveolin/eNOS and hsp90/eNOS interactions in either condition was examined. As shown in FIG. 5A (top left panel), the amount of caveolin immunoprecipitated by eNOS antibodies was not significantly altered by the statin treatment in CMECs (P>0.1, n=3), whereas it appeared dramatically decreased in atorvastatin-treated AoECs (−73+/−7%, P<0.01, n=3).

To better appreciate the absolute amounts of eNOS interacting with caveolin in AoECs, reverse IP was also performed, using caveolin antibodies to immunoprecipitate eNOS, and compared the eNOS immunoblot signal with that resulting from the direct IP using eNOS antibodies. Densitometric analysis of immunoblots as presented in FIG. 5A (bottom panels) revealed that, whereas in CMECs <5% of the cell eNOS pool was bound to caveolin (and not altered by the statin treatment), 44+/−8% (n=6) of eNOS was found in the caveolin immunoprecipitate from AoEC extracts; importantly, this amount was reduced to 8+/−5% (n=6) when AoECs had been exposed to atorvastatin for 5 days (see FIG. 5A).

Hereunder is proven that atorvastatin decreases the abundance of caveolin in macrovascular but not microvascular Ecs.

As depicted in FIG. 5A (top right), the extent of caveolin recovered from the caveolin IP was reduced in AoECs exposed to atorvastatin. Therefore, the effects of statin exposure on the absolute amounts of caveolin in AoECs and CMECs was examined. FIG. 5B confirmed that the AoEC pool of caveolin was reduced by 26+/−5% (n=3, P<0.01) in the presence of atorvastatin, whereas, as expected, the 3-hour exposure of CMECs to the statin did not alter the abundance of caveolin. To further assess whether this difference was dependent on the cell type (eg, macro- versus microvascular) or the experimental model (eg, isolated versus outgrowing ECs), the effects of statins on caveolin expression in isolated bovine aortic ECs (AECs) and in outgrowing ECs recovered from human coronary microvessels (CoMECs) cultured in Matrigel was also evaluated. FIG. 5B shows that the endothelial bed was critical because both outgrowing and isolated aortic ECs revealed a significant decrease in caveolin abundance after statin treatment, whereas microvascular outgrowing (CoMECs) and isolated cells (CMECs) did not any alteration in caveolin expression (see also FIG. 5C).

Remarkably, when the data presented in FIG. 5B were normalized to the nurnber of cells (see FIG. 5C), the pool of caveolin in AoECs and AECs appeared ˜6- to 10-fold lower than that of microvascular ECs (CMECs and CoMECs), which therefore are likely to be insensitive to small changes in the abundance of the scaffolding protein. Conversely, the cellular pool of eNOS (and of hsp90) was not significantly different between the different cell types tested (not shown). Importantly, supplementation of the culture medium with mevalonate (the immediate downstream metabolite of 3-hydroxy-3-methylglutaryl coenzyme A [HMGCOA] reductase) blocked the statin-induced reduction in caveolin abundance in AECs and AoECs and the associated proangiogenic effect (not shown).

Because the difference in the absolute amounts of caveolin in the cell appeared critical for the effects of statins on eNOS activity, it was further sought to determine whether another function associated with caveolin was modified by the statin treatment exclusively in cells with the lower pool of caveolin. The free cholesterol (FC) efflux from AECs and CMECs was compared by measuring the decrease in (radiolabeled) cell cholesterol after the addition of 20% serum to the cell-bathing medium. Under these conditions, the rate of FC efflux was significantly lower in low-caveolin-expressing AECs than in CMECs (−15+/−2% versus −33+/−3% of total [³H]cholesterol content after 4 hours, respectively; see FIG. 5D, open symbols). Remarkably, when ECs were first exposed to statins, FC efflux was not altered in CMECs but was almost completely blocked in AECs (P<0.01; n=3; see FIG. 5D, closed symbols), in agreement with the reduction in caveolin abundance observed in these cells on statin exposure.

Hereunder is proven that atorvastatin increases the extent of the hsp90/eNOS interaction and eNOS phosphorylation in Ecs.

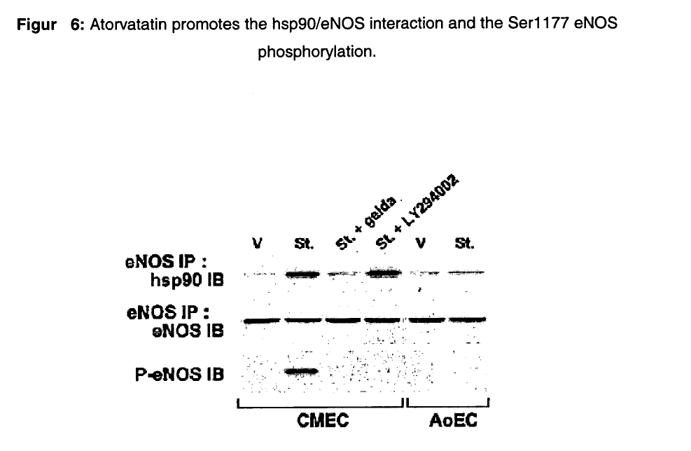

The analysis of the amount of hsp90 immunoprecipitated by eNOS antibodies revealed that the hsp90/eNOS interaction was increased by ˜5-fold in CMECs exposed for 3 hours to atorvastatin (FIG. 6, top). Furthermore, a 30-minute preincubation with 1 μg/mL geldanamycin (known to block the hsp90 chaperone function) led to a nearly complete blockade of both the recruitment of hsp90 to eNOS, at least under performed detergent conditions (FIG. 6, top), and the reorganization of CMECs in tubes (not shown).

The modulation of eNOS phosphorylation by statins was examined using anti-phospho-Ser1177 eNOS antibodies. FIG. 6 (bottom) shows that exposure of CMECs to atorvastatin did produce eNOS phosphorylation in a PI(3)K-dependent manner because the PI(3)K inhibitor LY294002 completely blocked the statin-induced phosphorylation of eNOS. Interestingly, although LY294002 did not prevent the hsp90/eNOS interaction, geldanamycin blocked the phosphorylation of eNOS, suggesting that in CMECs, hsp90 binding to eNOS is a prerequisite for statin-induced phosphorylation of eNOS.

In AoECs, no effect of statin treatment was observed on the phosphorylation of eNOS. However, this appeared to be dependent on the experimental model; the 48-hour period in the absence of atorvastatin (after the 5-day treatment with the drug) precluded the IB detection of the phosphorylated form of eNOS. Additional experiments showed, that an uninterrupted exposure of outgrowing AoECs (or isolated AECs) to the statin did produce a significant increase in the amounts of phospho-eNOS and hsp90/eNOS interaction (not shown).

Hereunder is proven that hsp90 is required for an efficient phosphorylation of eNOS by Akt upon statin exposure.

In a next step, the molecular mechanisms governing the statin-induced phosphorylation of Akt and eNOS and the subsequent tube formation by modulating hsp90 and caveolin abundance was characterized. The need for time course studies and various pharmacological treatments prompted to use the more easily isolatable and tractable HUVECs (instead of CMECs).

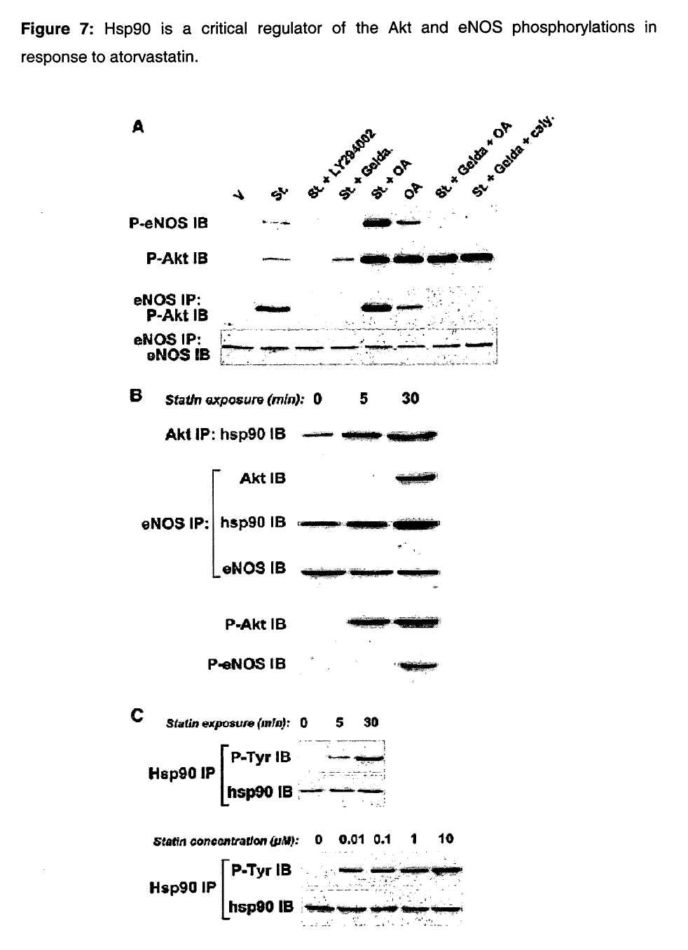

First it was verified that statins produced the rapid phosphorylation of Ser1177 eNOS in HUVECs and that this process was PI(3)K/Akt-dependent using LY294002 (see FIG. 7A). Interestingly, whereas IB with anti-phospho-Ser473 Akt antibodies revealed only a weak signal on statin treatment, the presence of a robust phospho-Akt signal in the eNOS immunoprecipitate was identified (FIG. 7A).

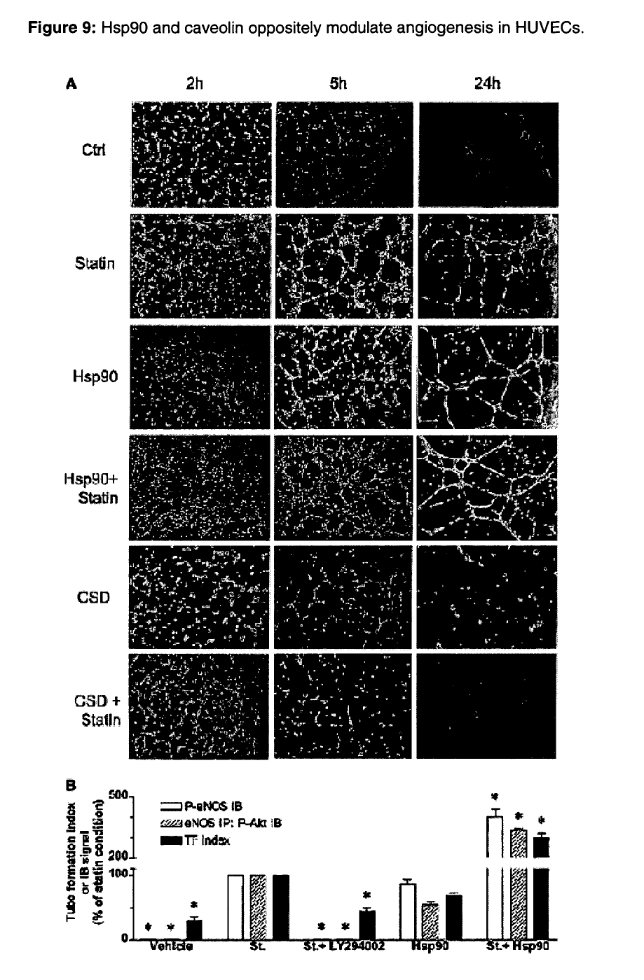

To further characterize the role of hsp90 in mediating statin-induced Akt-mediated angiogenic effects in HUVECs, the use of geldanamycin and okadaic acid, a phosphatase (PP2) inhibitor which has been reported to prevent Akt dephosphorylation was combined. FIG. 7A indicates that the statin-induced Akt-dependent phosphorylation of eNOS was hsp90-mediated because it was inhibited by geldanamycin. Furthermore, in the presence of okadaic acid, the (basal) level of Akt phosphorylation was dramatically increased and the statin-induced phosphorylation of eNOS was increased by ˜3-fold (compare the okadaic acid versus okadaic acid+statin conditions in FIG. 7A). Importantly, the simultaneous preincubation with geldanamycin and okadaic acid did not change the extent of Akt phosphorylation but completely repressed the phosphorylation of eNOS, indicating that the phosphorylation of eNOS is not strictly dependent on the level of Akt phosphorylation but instead on the level of hsp90 bound to eNOS. To confirm this finding, the interaction between phosphorylated Akt and eNOS was quantified by co-IP. FIG. 7A shows that the extent of phospho-Akt/eNOS interaction matched the level of eNOS phosphorylation (see also solid bars in FIG. 9B); ie, both were repressed by LY294002 and geldanamycin whereas they were promoted by okadaic acid+statin. Similar results were obtained using calyculin, a phosphatase inhibitor with a larger spectrum (PP1+PP2) (FIG. 7A).

Hereunder is proven that atorvastatin independently promotes the Akt/hsp90 and eNOS/hsp90 interactions and stimulates the hspgo tyrosine phosphorylation.

Although the data presented in FIG. 7A documented that the phosphorylation of eNOS on Ser1177 depends on the ability of Hsp90 to recruit Akt in the eNOS complex, the exact nature of the eNOS multicomplex induced by statins was not clear. Therefore, the time course of the Akt/hsp90, eNOS/Akt, and eNOS/hsp90 interactions was examined in co-IP experiments. As shown in FIG. 7B (top), a 5-minute exposure to atorvastatin was sufficient to promote the interaction of hsp90 with both Akt and eNOS but insufficient to promote the association of Akt with eNOS in HUVECs. In agreement with these observations, the 5-minute incubation with atorvastatin did not promote eNOS phosphorylation, whereas it was sufficient to induce Akt phosphorylation as shown in IB experiments (FIG. 7B, bottom). As expected, longer (30 minutes) exposure to the statin produced eNOS phosphorylation and the association of Akt-hsp90-eNOS in a trimeric complex, as detected by mutual co-IP (FIG. 7B, last lanes). Of note, the extent of this mutual interaction reached its maximum after 1 to 2 hour(s) of atorvastatin treatment.

The direct effect of statins on the ability of hsp90 to act as a scaffold (independently for Akt and eNOS) incited to examine whether hsp90 was itself modified by phosphorylation. FIG. 7C shows that atorvastatin rapidly and dose-dependently induced the tyrosine phosphorylation of hsp90.

Hereunder is proven that hsp90 overexpression potentiates the Akt-mediated phosphorylation of eNOS in response to statins.

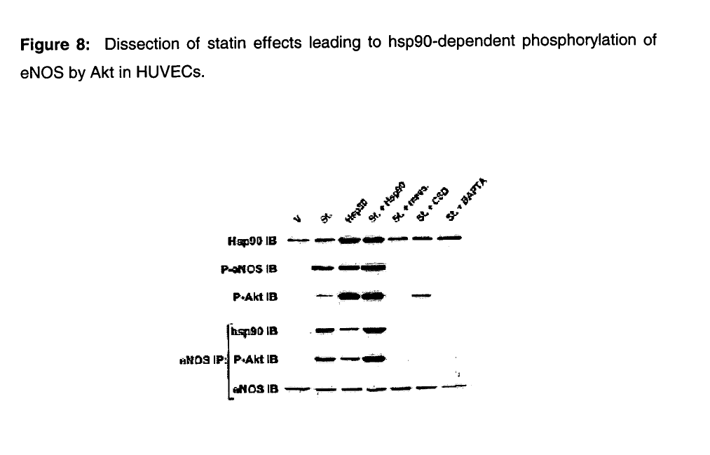

Having documented that modulating the integrity of the hsp90/eNOS interaction directly affected the statin-induced phosphorylation of Ser1177 eNOS, the role of hsp90 was further examined by increasing the abundance of this chaperone protein in HUVECs by heterologous expression. IB experiments revealed that hsp90 overexpression and statin exposure separately led to a slight increase in eNOS phosphorylation (FIGS. 8 [left] and 9B [open bars]). When both hsp90 overexpression and the statin treatment were combined, the level of eNOS phosphorylation was synergistically increased together with the extent of the eNOS/hsp90 association (see FIGS. 8 and 9B).

FIG. 8 (left) also shows that a phospho-Akt signal could be detected in the eNOS IP from statin-treated cells, and importantly that the enrichment in phospho-Akt was increased >3-fold when statin-treated cells also expressed recombinant hsp90 (see also FIG. 9B, hatched bars). This potentiation of the effect of statin treatment by hsp90 over-expression was observed, whereas the level of phospho-Akt was not significantly higher than under the conditions of hsp90 overexpression alone (ie, in the absence of atorvastatin), thereby emphasizing the critical role of hsp90 in recruiting Akt in the vicinity of eNOS to allow enzyme phosphorylation.

Hereunder is illustrated that hsp90-dependent phosphorylation of eNOS after statin treatment is sensitive to mevalonate and Ca²-chelation and is blocked by the CSD peptide.

The mechanism by which statins impact on the hsp90-dependent regulation of eNOS activation was further dissected. First, the addition of mevalonate was sufficient to reverse the stimulatory effect of atorvastatin on eNOS/hsp90 interaction as well as on the phosphorylation of both eNOS and the fraction of Akt associated with eNOS (FIG. 8, right).

The effects of synthetic peptides derived from the CSD introduced by reversible permeabilization in HUVECs before plating on Matrigel was also examined. Immunoblot analyses revealed that cell loading with CSD peptides prevented the association of eNOS with hsp90 and Akt and blocked the phosphorylation of eNOS, even though it did not influence the phosphorylation of Akt (FIG. 8, right). In a next step, changes in intracellular calcium were analyzed and its effect on the statin-induced Akt/eNOS activation pathway was studied. FIG. 8 shows that when the calcium chelator BAPTA-AM was added to the cells 30 minutes before the treatment with statin, the interaction of eNOS with hsp90 and its phosphorylation were abrogated. Interestingly, the phosphorylation of tyrosine phosphorylation of Akt was also blocked by BAPTA treatment, suggesting that under the performed experimental conditions, Akt phosphorylation by statins is a calcium-dependent mechanism or, at least, is sensitive to changes in [Ca²⁺]i.

Hereunder evidence is shown proving that hsp90 and caveolin oppositely modulate the atorvastatin-induced endothelial network organization.

In a next step, the influence of hsp90 overexpression or CSD peptide loading (see FIG. 8) on the increase or decrease the proangiogenic effects of statins, respectively, in the model of HUVECs cultured on Matrigel was analyzed.

FIG. 9A shows that atorvastatin (0.1 μmol/L) stimulated tube formation in HUVECs with kinetics similar to those observed with CMECs (see FIG. 4) and, importantly, that the heterologous expression of hsp90 significantly potentiated this proangiogenic effect for at least 24 hours under the performed assay conditions (see FIGS. 9A and 9B [solid bars]). Of note, as already observed with the CMECs, the use of LY294002 prevented HUVEC reorganization induced by statins and/or by hsp90 overexpression (see FIG. 9B). Conversely, the phosphatase inhibitor okadaic acid stimulated basal and statin-induced tube formation (not shown). Furthermore, when HUVECs were first loaded with CSD peptides, atorvastatin treatment was unable to stimulate tube formation (see FIGS. 9A and 9B), whereas the use of a scrambled CSD peptide was without effect on the proangiogenic effects of statins (not shown).

This example identifies hsp90 as a key player in the proangiogenic action of statins and underscores the importance of caveolin as a regulator of NO-dependent angiogenesis.

One of the major findings of this example is that the capacity of statins to reduce the extent of the inhibitory caveolin/eNOS interaction, and thereby to stimulate NO-mediated angiogenesis, is dependent on the EC type. Although caveolin overexpression or caveolin peptide leads to the inhibition of NO-dependent processes in virtually any type of cells expressing eNOS, the effects of statins on the caveolin/eNOS interaction vary from one EC type to another.

The invention teaches that the reduction in caveolin abundance induced by statin treatment accounts for (part of) the proangiogenic effects of these hypolipidemic drugs exclusively in ECs with a low pool of caveolin. Accordingly, in aortic ECs, the low cellular pool of caveolin is very sensitive to the statin treatment, whereas in microvascular ECs (either isolated or outgrowing), the 6- to 8-fold higher pool of caveolin is not responsive to the statin exposure. Likewise, the effects of statins on the rate of FC efflux from serum-stimulated ECs appear dependent on the abundance of caveolin. Atorvastatin failed, indeed, to modulate the FC efflux in CMECs (which have a high caveolin pool), whereas in statin-exposed aortic ECs, a nearly complete blockade of the cholesterol efflux was observed under the performed assay conditions. Interestingly, this observation, ie, the much greater abundance of caveolin in the microcirculation relative to macrovessels, is consistent with NO being the major vasodilator in the conduit arteries compared with the microvessels in which endothelium-derived hyperpolarizing factors may predominate.

Moreover, whereas in aortic ECs a high proportion of eNOS interacts with caveolin, in the coronary microvascular ECs, the caveolin/eNOS interaction is limited to a few percent of total eNOS, and the activity of eNOS is therefore intrinsically less conditional on the statin effects on caveolin abundance. Therefore, the conjunction of this parameter, eg, the extent of the interaction between eNOS and caveolin, with the extent of the caveolin pool, determines the effects of statins on the stoichiometry of the caveolin/eNOS interaction and the subsequent NO-mediated angiogenic effects.

The second major finding of this example of the present invention is that the capacity of statins to stimulate the formation of capillary-like structures can be blocked by inhibiting the function of hsp90 (by the use of geldanamycin) and conversely, promoted after hsp90 overexpression.

Indeed, this example provides evidence that although Akt-dependent phosphorylation of Ser1177 eNOS ultimately accounts for the NO-mediated proangiogenic effects of statins, hsp90 is the control point for this effect. Thus, overexpression of hsp90 by itself (without statin treatment) led to an increased phosphorylation of Akt and eNOS, and to the formation of capillary-like structures. Also, statin treatment and hsp90 overexpression synergistically increased both the level of Ser1177 eNOS phosphorylation and the stability of the endothelial tube reorganization (see FIG. 8); under the performed experimental conditions, changes in the phosphorylation status of Thr495 (another eNOS phosphor-ylatable residue) do not appear to account for the statin effects. Finally, this example shows that whereas the phosphatase inhibitor okadaic acid (and calyculin) stimulated Akt phosphorylation, the blockade of the hsp90/eNOS interaction by geldanamycin suppressed the ability of statins to promote eNOS phosphorylation and tube formation (whereas it had no effect on Akt phosphorylation [see FIG. 7A]).

Furthermore, the findings of example 3 are consistent with the following sequence of events: (1) statin treatment stimulates the interactions of hsp90 with eNOS and Akt (independently), (2) hsp90 acts as a scaffold bridging activated Akt and eNOS, and (3) eNOS is phosphorylated on Ser1177 by neorecruited Akt. In fact, present invention revealed that statins by targeting hsp90 have a double beneficial effect leading to the potentiation of the activation of both eNOS and Akt. Altogether, these findings emphasize the critical role of hsp90 in driving the statin effects and particularly those leading to angiogenesis.

The results of example 3 of present invention also shed some light on the upstream mechanisms leading to the hsp90 binding to eNOS and Akt after statin exposure. Indeed, the promoting effects of statins on the eNOS/hsp90 interaction were reversed by mevalonate, thereby attesting to the implication of the HMGCoA reductase and probably of the isoprenoid synthesis. Moreover, the effect of statins on the recruitment of hsp90/phospho-Akt to form a multicomplex with eNOS appeared closely dependent on changes in [Ca²⁺]ᵢ as revealed by the inhibitory effects of the calcium chelator BAPTA in the present example (see FIG. 9). By antagonizing the Ca²⁺/calmodulin-mediated activation of eNOS by loading HUVECs with synthetic peptides corresponding to the CSD sequence, both the statin-induced tube organization and the eNOS multicomplex formation could be completely prevented. Finally, hsp90 was found to be rapidly and dose-dependently tyrosine phosphorylated on statin exposure (FIG. 7C), confirming the direct effect of the statin on the chaperone protein.

In summary, the caveolin/eNOS and hsp90/eNOS interactions was identified as key pharmacological targets to modulate NO-dependent angiogenesis. This supports the therapeutic potential of statins but also of other modulators of hsp90 and/or caveolin to promote neovascularization, particularly in the context of ischemic diseases.

In Example 4 evidence is shown that hsp90 ensures the transition from the Early Ca²⁺-dependent to the late phosphorylation-dependent activation of the endothelial nitric-oxide synthase in vascular endothelial growth factor-exposed endothelial cells.

Vascular endothelial growth factor (VEGF) exerts its angiogenic effects partly through the activation of endothelial nitric-oxide synthase (eNOS). The interplay between these different mechanisms were examined in VEGF-exposed endothelial cells. This example of present invention documents that hsp90 binding to eNOS is, in fact, the crucial event triggering the transition from the Ca²⁺-dependent activation of eNOS to the phosphorylation-mediated potentiation of its activity by VEGF. Accordingly, it was shown that early VEGF stimulation first leads to the Ca²⁺/calmodulin disruption of the caveolin-eNOS complex and promotes the association between eNOS and hsp90. eNOS-bound hsp90 can then recruit VEGF-acti-vated (phosphorylated) Akt to the complex, which in turn can phosphorylate eNOS. Further experiments in transfected COS cells expressing either wild-type or S1177A mutant eNOS led to identify the serine 1177 as the critical residue for the hsp90-dependent Akt-mediated activation of eNOS. Finally, it was found that although the VEGF-induced phosphorylation of eNOS leads to a sustained production of NO independently of a maintained increase in [Ca²⁺]i, this late stage of eNOS activation is strictly conditional on the initial VEGF-induced Ca²⁺-dependent stimulation of the enzyme. These data establish the critical temporal sequence of events leading to the sustained activation of eNOS by VEGF and suggest new ways of regulating the production of NO in response to this cytokine through the ubiquitous chaperone protein, hsp90. In this example, a model of human EC in culture as well as the heterologous COS cell expression system to dissect the specific protein-protein interactions, their inter-dependence, and temporal sequence involved in the post-translational regulation of eNOS by VEGF.

Cell Culture was performed as follows: Freshly isolated human umbilical vein EC (HUVEC) were cultured to confluence in 100-mm dishes in a 2:1 mixture of M199/endothelial cell basal medium containing 10% serum; control experiments were also performed with myocytes isolated from neonatal mice as previously detailed (Feron, et al. (1998) J. Biol. Chem. 273, 30249-30254) and plated at 5×10 5 cells/ml. Serum-starved EC and myocytes were exposed for the indicated periods of time to VEGF (50 ng/ml) and insulin (12.5 milliunits/ml), respectively, in the presence or absence of various pharmacological modulators (30-60 min preincubation with 1 μg/ml geldanamycin (Invitrogen Life Technolo-gies) or 10 μM LY294002 (Sigma)).

The transfection was performed as follows: COS-7 cells were transfected with the different plasmid cDNAs using LipofectAMINE (Life Technologies Inc.) according to the manufacturers protocol; eNOS and Akt constructs were a gift from Dr. S. Dimmeler and the hsp90 cDNA was provided by Dr. W. C. Sessa. The expression of appropriate proteins was confirmed by Western blotting and an irrelevant plasmid encoding B-galactosidase was used as a control to maintain identical amounts of DNA in each transfection.

Immunoprecipitation, Immunoblotting, and Kinase Assay was performed as follows: EC were homogenized in a buffer containing phosphatase and protease inhibitors (Garcia-Cardena, G., et al. (1998) Nature 392, 821-824), and processed for immunoblotting or immunoprecipitation in a octyl glucoside-containing buffer as described previously (Feron, O., et al. (1998) J. Biol. Chem. 273, 3125-3128). Caveolin, eNOS, and hsp90 antibodies were from BD Transduction Labs, Akt and phospho-Akt antibodies from BD PharMingen, CaM antibody from Santa Cruz, and phospho-eNOS antibody from NEB Cell Signaling Technology. Akt activity was measured using a non-radioactive immunoprecipitation kinase assay kit (NEB Cell Signaling Technology).

Changes in [Ca²⁺]i were measurement using following assay conditions: EC plated on coverslips were incubated with 2 μM Fura-2/AM at 37° C. for 30 min and then extensively washed and placed in a temperature-controlled perfusion chamber for 15 min. Cells were observed with a Zeiss Axiovert 100 microscope in the epifluorescence mode using a long pass filter cut-off at 510 nm and a dichroic at 405 nm. Alternating wavelength excitation of 340 and 380 nm was provided by a motorized filter wheel. The image pairs monitored by the lonoptix Myocam camera were processed by the IonWizard software (IonOptix, Milton, Mass.) and are presented as the fluorescence ratio (340/380 nm), a direct index to the [Ca²⁺]i. In some experiments, Fura-2-loaded cells were preincubated with the intracellular calcium chelator BAPTA/AM (20 μM). After rinsing coverslips, EC were left to recover for 5 min before VEGF stimulation.

Nitrite was Detected as follows: Quantitative analysis of nitrite (NO₂⁻) was used as an index of NO production. Briefly, aliquots of the medium bathing intact EC were collected at the time intervals corresponding to either 0-2 or 5-30 min of VEGF exposure. For the latter period, the medium bathing the VEGF-stimulated cells for the first 5 min was discarded and replaced by fresh VEGF-containing medium for the next 25 min. In COS cells experiments, NO₂⁻ accumulation in the interval time of 44 to 48 h after transfection was measured. Acidic iodide was used to convert NO₂⁻ to NO that was electrochemically measured with an NO-selective microsensor (WPI), as recommended by the manufacturer; adequate controls using either vehicle or NOS inhibitors were routinely performed in parallel. Data are normalized for the amount of protein in the dish, and are presented for convenience as mean +/−S.E. Statistical analyses were made using Student's t test or one-way ANOVA where appropriate.

This example illustrates that endothelial cell exposure to VEGF promotes caveolin-eNOS complex disruption and hsp90/eNOS Interaction.

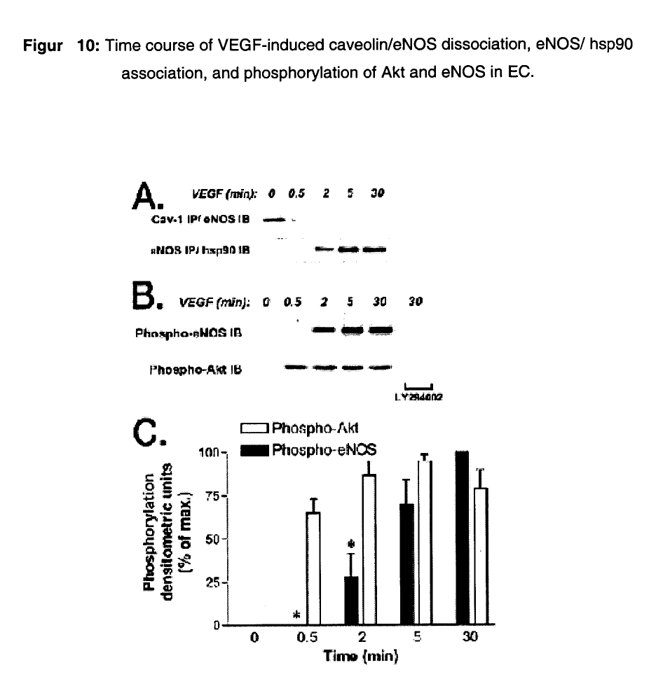

Following exposure to VEGF (50 ng/ml) for increasing periods of time (0, 0.5, 2, 5, and 30 min), EC were collected, lysed, and cell extracts used for immunoprecipitation as follows. First, extracts were immunoprecipitated with caveolin antibodies and the immuno-precipitation immunoblotted with eNOS antibodies (FIG. 10A, top). The results showed that the fraction of eNOS associated to caveolin in basal conditions rapidly dissociated from caveolin when EC were exposed to VEGF. In fact, the dissociation was complete in the first 2 min of VEGF exposure and was maintained for at least 30 min in the presence of VEGF. Experiments were then performed to determine the time course of hsp90 interaction with eNOS. FIG. 10A (bottom) shows that, in these experimental conditions, by using eNOS antibodies, hsp90 is able to co-immunoprecipitate after a minimum of 2 min of cell exposure to VEGF, e.g. after the dissociation of the caveolin-eNOS complex.

Hereunder evidence is presented that Akt phosphorylation precedes eNOS phosphorylation in EC exposed to VEGF.

In a parallel series of experiments, the time course of both Akt and eNOS phosphorylation was examined in VEGF-stimulated EC using antibodies directed against phosphorylated Ser 473 and Ser 1177 in Akt and eNOS, respectively. Maximal eNOS phosphorylation occurred after at least 5 min of VEGF exposure (FIG. 10B, top) whereas the stimulatory effect of VEGF on Akt phosphorylation was already observed at 30 s in the performed experimental conditions (FIG. 10B, bottom); densitometric analysis of these observations is presented in FIG. 10C. To further assess if the VEGF/Akt signaling cascade accounted for the observed eNOS phosphorylation in the performed experimental conditions, the PI3K inhibitor LY294002 known to prevent phosphorylation and activation of the downstream effector Akt was used. A 1-h preincubation with LY294002 (10 μM) completely prevented both Akt and eNOS phosphorylation upon 30 min VEGF stimulation (FIG. 1B (right)).

Evidence is given hereunder showing that eNOS recruits Akt through a mutual hsp90 interaction.

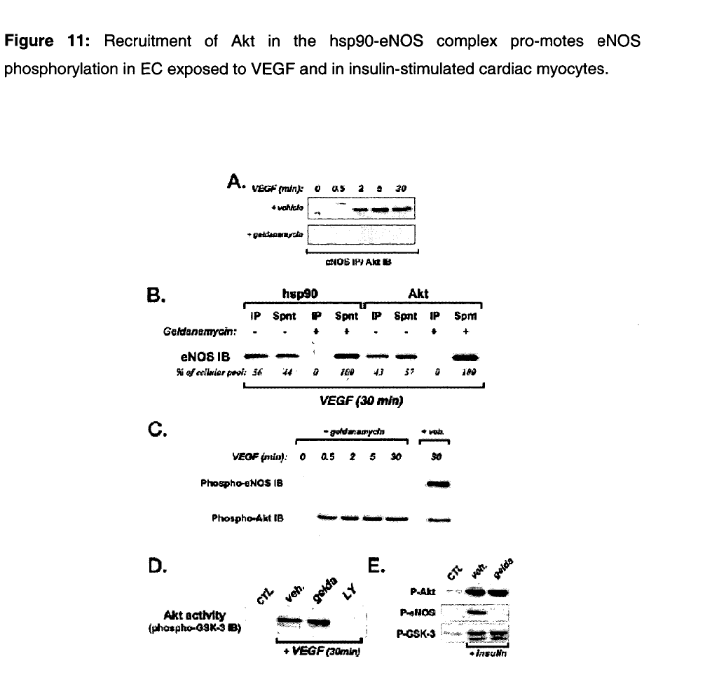

It was further analyzed whether eNOS and Akt interact together in eNOS/Akt co-immunoprecipitation experiments. Interestingly, as illustrated in FIG. 11A (top), the co-immunoprecipitation of Akt with eNOS was consistently observed with a time course in agreement with the observed pattern of eNOS phosphorylation (see FIG. 10B) and hsp90/eNOS interaction (see FIG. 10A); using anti-phospho-Akt antibodies, it was verified that the eNOS-bound Akt was the activated (phosphorylated) form of the kinase (not shown). Based on this correlation in the kinetics, these different events were examined and tested whether they were directly related. EC were pre-exposed for 30 min, before VEGF stimulation, to geldanamycin (1 μg/ml), an agent known to inhibit hsp90 function, and corresponding lysates were submitted to immunoprecipitation with eNOS antibodies. In these conditions, hsp90 was no longer detectable in eNOS immunoprecipitation (not shown) and neither was Akt (FIG. 11A, bottom).

In an attempt to appreciate the proportion of eNOS involved in the geldanamycin-sensitive recruitment of Akt, the reverse co-immunoprecipitation assay was further developed: hsp90 and Akt antibodies were used to imunoprecipitate eNOS (which was detected by immunoblotting) and the supernatants of these immunoprecipitations were checked for residual eNOS presence; for this purpose, the immunoprecipitated supernatants were submitted to a second immunoprecipitation with eNOS antibodies. Since both hsp90/eNOS (FIG. 10A) and Akt/eNOS co-immunoprecipitation (FIG. 11A) appeared maximal after 5 min VEGF exposure, the immuno-precipitation was performed from extracts of EC pre-exposed for 5 min to VEGF in the presence or absence of geldanamycin. FIG. 11B (first and second lanes) shows that approximately the same amount of eNOS that was immunoprecipitated by hsp90 antibodies was found in the supernatant of this immunoprecipitation. A similar pattern was found for the Akt/eNOS immunoprecipitation (FIG. 11B, fifth and sixth lanes), indicating that around half of the total eNOS pool was engaged in hsp90 and Akt interaction in the performed assay conditions. The geldanamycin pretreatment did not lead to any detectable amount of eNOS immunoprecipitated either by hsp90 or Akt antibodies (FIG. 11B, third and seventh lanes); all the pool of eNOS was found in the supernatants of these immunoprecipitations, as detected by immunoblotting after a second immunoprecipitation with eNOS antibodies (FIG. 11B, fourth and eighth lanes).

The example illustrates hereunder that eNOS phosphorylation requires the hsp90-mediated recruitment of activated Akt.

To further test the present hypothesis that hsp90/eNOS interaction is necessary for Akt phosphorylation of eNOS, Akt and eNOS phosphorylation patterns were also examined in the presence of geldanamycin. From these experiments it was observed that although Akt phosphorylation was detectable as soon as 30 s after VEGF addition despite the presence of geldanamycin (FIG. 11C, bottom), no phospho-eNOS signal was detectable even upon prolonged VEGF exposures (FIG. 11C, top) in striking contrast with the control condition (FIG. 11C, right). It was also verified that geldanamycin treatment had no significant effect on the time course of caveolin/eNOS dissociation (not shown).

To exclude a direct effect of geldanamycin on Akt, Akt activity was measured in EC exposed for 30 min to VEGF in the presence or absence of geldanamycin. After selective im-munoprecipitation of Akt from the corresponding lysates, immunoprecipitates were incubated with purified GSK-3 and Akt-induced phosphorylation of GSK-3 was evaluated by im-munoblotting using specific anti-phospho-GSK-3 antibody. FIG. 11D shows that the 30-min VEGF-stimulated Akt activity was not altered by the pretreatment of cells with geldanamycin, whereas not surprisingly, the P13K inhibitor LY294002 abrogated the Akt activation in VEGF-exposed EC. In a next step, it was sought whether the inhibitory effect of geldanamycin on eNOS phosphorylation could be reproduced in another cell type and upon another agonist stimulation. Accordingly, neonatal mouse cardiac myocytes were exposed to insulin (a known activator of Akt in myocytes) for 5 min with or without geldanamycin and examined the phosphorylation status of Akt and eNOS. FIG. 11E shows that while the pretreatment with geldanamycin (1 μg/ml) did not alter the insulin-induced Akt activation, it completely abolished eNOS phosphorylation. Myocytes show native expression of GSK-3 and were used to determine whether geldanamycin could influence the phosphorylation of this other Akt substrate. FIG. 11E (lower panel) shows that the insulin-induced increase in GSK-3 phosphorylation was not altered by geldanamycin. Of note, the stimulatory effect of insulin on Akt, eNOS, and GSK-3 phosphorylation was blocked by preincubation of myocytes with 50 μM LY294002 (not shown).

Hereunder it is shown that hsp90 facilitates Akt-dependent NO production through eNOS phosphorylation on Ser 1177.

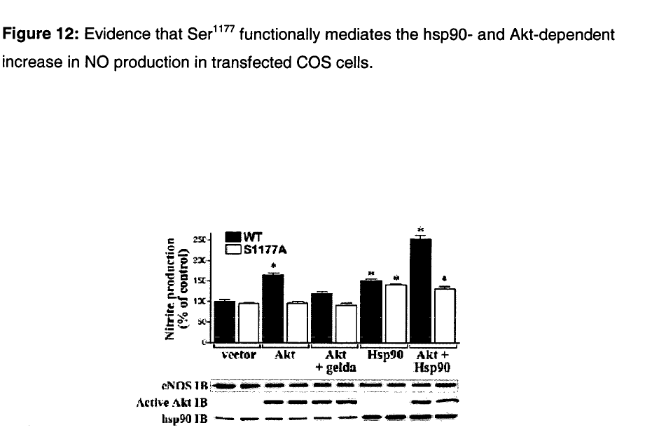

Transfection experiments were carried out to evaluate the effect of geldanamycin treatment and hsp90 overexpression in COS cells expressing either wild-type eNOS or S1177A eNOS (construct in which the serine 1177 is mutated in alanine). First COS cells were co-transfected with active Akt (T308D/S473D), a significant increase in nitrite production was observed in WT-eNOS-expressing cells (+64+/−4%) whereas no change in eNOS activity was detected with the non-phosphorylable S1177A eNOS cells (FIG. 12, see bars 3 and 4). Interestingly, when COS cells were pretreated for 30 min with geldanamycin, around 70% of the Akt-dependent increase in nitrite production in WT-eNOS cells was blocked (FIG. 12, bar 5). In another series of experiments, the cellular pool of hsp90 was increased by 2-fold (see FIG. 12, lower panel) by co-transfecting hsp90 cDNA either with WT- or S1177A-eNOS constructs. As shown in FIG. 12 (bars 7 and 8), the consecutive increase in nitrite production (+49+/−4 and +40+/−3%, respectively) was similar in both conditions. By contrast, when active Akt (T308D/S473D) was also present in the system, a synergistic increase of 152+/−8% over basal level of nitrite production was observed in cells expressing WT-eNOS (FIG. 12, bar 9), whereas no further change (over the hsp90-alone condition) was observed in S1177A eNOS cells (FIG. 12, compare bars 8 and 10); preincubation with geldanamycin blocked 70-95% of the recombinant hsp90- and hsp90/Akt-mediated increase in NO production (not shown). The lower panel of FIG. 12 shows the immunoblots for eNOS (WT and S1177A), Myc-tagged Akt and hsp90 (recombinant+endogenous).

In this paragraph, evidence is given showing that late eNOS phosphorylation is dependent on the early VEGF-evoked increase in [Ca²⁺]i.

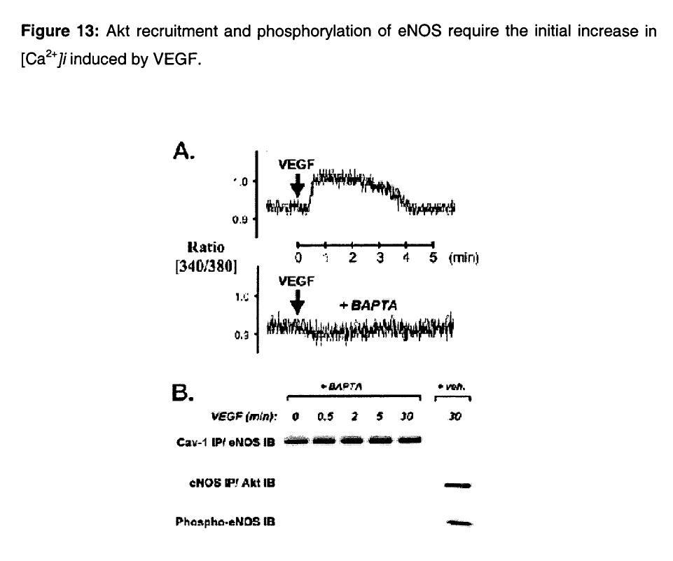

In a next step the calcium dependence of the Akt/hsp90 recruitment and eNOS phosphorylation process was studied by preincubating EC with the intracellular calcium chelator BAPTA before exposure to VEGF. FIG. 13A shows the pattern of changes in [Ca²⁺]i in EC exposed to VEGF in the absence and presence of 20 μM BAPTA-AM. VEGF exposure led to a slightly delayed calcium transient peaking after 1 min and slowly returning to basal levels in agreement with the reported phospholipase C-γ-dependent calcium release from internal stores. In the presence of BAPTA, the VEGF-evoked calcium transient was completely abrogated, indicating that under these experimental conditions, any further change in eNOS activity may be considered as independent of changes in measurable [Ca²⁺]i.

In conditions where BAPTA blocked the VEGF-induced in-crease in [Ca²⁺]i a complete inhibition of caveolin/eNOS dissociation was observed as reflected by the consistent amount of eNOS co-immunoprecipitated with caveolin over time in response to VEGF exposure (FIG. 13B, top). Calcium clamping by BAPTA also prevented the VEGF-induced Akt/eNOS co-immunoprecipitation (FIG. 13B, middle) and eNOS phosphorylation (FIG. 13B, bottom). Of note, the hsp90/eNOS co-immunoprecipitation was also completely prevented by the use of BAPTA (not shown). Importantly, when using the 30-min VEGF exposure in the absence of BAPTA as a control condition, the dissociation of eNOS from caveolin was consistently observed, the eNOS/Akt interaction as well as eNOS phosphorylation (FIG. 13B, right).

Hereunder it is illustrated that hsp90 promotes the phosphorylation-dependent component of eNOS activation.

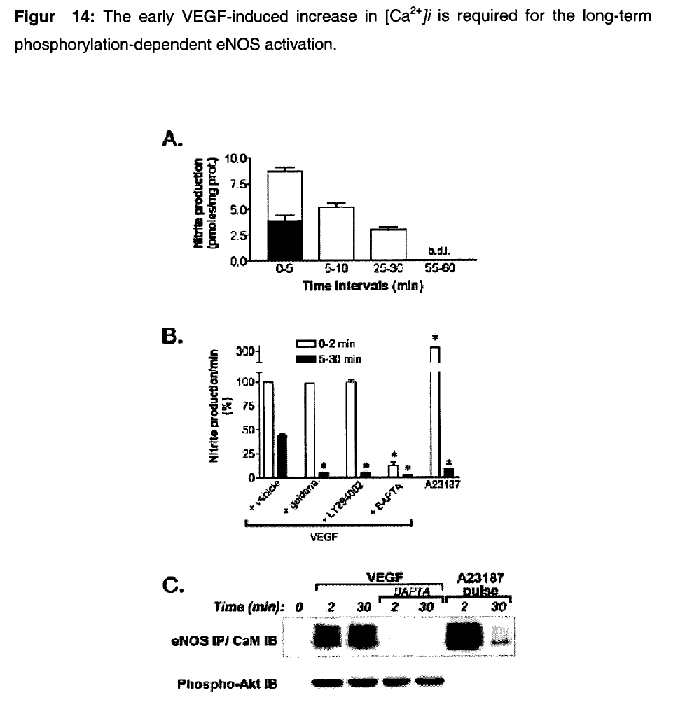

The activity of eNOS following VEGF stimulation was measured in the presence and absence of geldanamycin. Preliminary experiments revealed that the rate of NO₂⁻ produced in the first 5-min interval (0-5 min) after VEGF stimulation was ˜2-3-fold higher than in the following 30 min (FIG. 14A), in agreement with the 5-min duration of the VEGF-induced calcium transient (FIG. 13A). However, considering that the phosphorylation of eNOS was already maximal after 5 min (see FIG 10C), the levels of NO₂⁻ produced was compared in the 0-2- and 5-30-min intervals following VEGF addition in order to better distinguish between direct Ca²⁺/CaM-dependent and phosphorylation-mediated activation of eNOS.

As shown in FIG. 14B (where the nitrite production is normalized per min), geldanamycin pretreatment significantly inhibited the late VEGF-induced eNOS activation in agreement with the observed inhibition of interaction with hsp90 and Akt, whereas no significant effect on the early VEG F-induced eNOS activation was observed. Importantly, similar results were obtained using the P13K inhibitor LY294002 confirming that the effect of Akt phosphorylation on eNOS activation was limited to the late activation of the enzyme. Of note, the basal level of [Ca²⁺]i and the profile of the VEGF-induced calcium transient were not altered by pretreatments with geldanamycin or LY294002 (not shown).

In a next step, the calcium dependence of the early and late activation of eNOS was determined by measuring the VEGF-induced NO₂⁻ production in the presence of the intracellular calcium chelator BAPTA. As shown in FIG. 14B, preincubation with 20 μM BAPTA-AM, in conditions similar to those used in FIG. 13A, was sufficient to block not only the early (0-2 min) but also the late eNOS activation (5-30 min). Importantly, when BAPTA was added after the early phase of enzyme activation (at t=5 min), no significant reduction in the level of eNOS activation was observed for the 5-30min period (versus vehicle condition; not shown). Finally, the calcium ionophore A23187 was used to examine whether a temporary increase in [Ca²⁺]I similar to the initial Ca²⁺ rise evoked by VEGF was sufficient to produce both the early and late activation of eNOS. Accordingly, cells were incubated in the presence of 1 μM A23187 for 2 min and then extensively washed and placed in Ca²⁺-free medium containing 1 mM EGTA; in these conditions, [Ca²⁺]I increased upon A23187 exposure and returned to its basal level with a time course similar to that observed with VEGF (not shown). FIG. 14B shows that while the A23187 pulse led to an increase in early NO₂ production (0-2 min) amounting to ˜3-fold the level obtained with VEGF, the long-term A23187-stimulated NO₂⁻ production was significantly lower than in VEGF-exposed cells.

In order to further dissect the calcium dependence of the Akt-mediated phosphorylation of eNOS, the eNOS/CaM association was examined after 2 and 30 min VEGF exposure, i.e. at the maximum of the calcium transient and after the return of [Ca²⁺]i to its basal level, respectively. FIG. 14C (top) reveals that, in the experimental conditions as followed by present invention, CaM could be detected in the eNOS immune complex 2 min after VEGF stimulation and that this association persisted for at least 30 min. Interestingly, while pretreatment with BAPTA completely abrogated the CaM/eNOS interaction (FIG. 12B, top), the Akt phosphorylation was not altered by calcium chelation (FIG. 12B, bottom), thereby excluding an effect of calcium chelation on Akt activity to account for the blockade of late eNOS activation (see FIG. 14C). Finally, the temporary increase in [Ca²⁺]i induced by a short exposure to A23187 (see above) led to the early association between eNOS and CaM but not to the prolonged Akt activation, in agreement with the low level of NO production in the 5-30 min interval of exposure to VEGF (see last column in FIG. 14B).

The recent discovery of the regulatory phosphorylation of eNOS has considerably challenged the original assumption that NO production by this Ca²⁺/CaM-dependent enzyme primarily reflected changes in intracellular [Ca²⁺]. Even though several pharmacological studies had recently suggested the existence of a “Ca²⁺-independent” activation of eNOS (mainly induced by fluid shear-stress), the molecular basis for such a mechanism remained elusive. In this example of present invention it is documented that although eNOS phosphorylation (and sustained activation) by Akt persists well after the initial calcium transient induced by VEGF, both regulatory processes of NO production are intimately linked in endothelial cells. Moreover, it was shown that hsp90 binding to eNOS is required for this transition from the early Ca²⁺-dependent activation of eNOS to the late enzyme phosphorylation by Akt observed upon VEGF exposure.

In EC, the hsp90/eNOS interaction triggered by VEGF stimulation is a key event allowing eNOS phosphorylation by Akt and prolonged NO release is based on the following evidence. (i) Time course studies revealed that, chronologically, EC exposure to VEGF first led to eNOS dissociation from caveolin, a hallmark of the Ca²⁺/CaM-mediated activation of eNOS, and then to hsp90/eNOS interaction. The late occurrence of eNOS phosphorylation perfectly matched the time course of the hsp90/eNOS interaction. Furthermore, although the Akt phosphorylation was consistently observed in the first 30 s of EC exposure to VEGF, Akt association to eNOS could only be detected after 2 min of VEGF stimulation, e.g. concomitantly to hsp90/eNOS interaction. (ii) In transfected COS cells, hsp90 overexpression synergistically increased the extent of eNOS activation by Akt. Furthermore, these data clearly identified serine 1177 as the critical residue within the eNOS sequence accounting for the hsp90-dependent Akt-mediated activation of the enzyme. (iii) The use of the pharmacological hsp90 inhibitor, geldanamycin, allowed us to further demonstrate the obligatory and specific role of hsp90 in the eNOS phoshorylation process. Indeed, geldanamycin completely blocked the Akt recruitment and consecutive eNOS phosphorylation and activation whereas it did not interfere with Akt activation, Akt binding to hsp90, nor phosphorylation of another Akt substrate (see FIG. 11E)).

Presented data also shed some light on the Ca²⁺ dependence of both early and late eNOS activation. The experimental data indicate that the concomitant occurrence of both signaling cascades leads to the potentiation of NO release in VEGF-exposed EC. Thus, the early VEGF-induced calcium transient is obligatory to promote the efficient binding of CaM to the enzyme (see FIG. 14C) and the short-term burst of NO release independently of eNOS phosphorylation by Akt (see FIG. 14B). Importantly, the present invention also revealed that the CaM/eNOS association, that persists even though [Ca²⁺]i has returned to baseline (see FIGS. 13A and 14C) is critical for the late phase of enzyme activation. Indeed, the calcium chelator BAPTA, by preventing the VEGF-induced CaM/eNOS association, blocked not only the early phase of eNOS activation but also the late enzyme phosphorylation and prolonged NO pro-duction. However, although the VEGF-induced [Ca²⁺]/increase appears to be necessary for both aspects of eNOS activation, the only calcium transient is not sufficient to promote the prolonged NO release since no long-term eNOS activation was observed by mimicking a temporary increase in [Ca²⁺]i by a sequential protocol of cell exposure to the calcium ionophore A23187 and Ca²⁺-free medium. These data still do not exclude that in the case of G-protein-coupled receptors that mobilize [Ca²⁺]i in EC to a much larger extent than VEGF, the initial rise in calcium could directly lead to Akt phosphorylation through the activation of PI3K or the CaM-de-pendent protein kinase kinase. Of note, other kinases than Akt, such as the CaM-dependent protein kinase II or the mitogen-activated protein kinase, have been recently reported to promote the phosphorylation-dependent activation of eNOS following G-protein-coupled receptor stimulation.

The present data emphasize the role of hsp90 as a major control point to integrate opposing properties of eNOS (coupled versus uncoupled enzyme activity) or to bridge early and late (independent) signaling cascades leading to the acute and pro-longed NO release. While the ATP/ADP state of hsp90 seems to determine its modulatory effect on eNOS-derived NO production, the molecular details of this protein-protein interaction remain unclear at this time. Interestingly, the time course of the association of hsp90 with eNOS paralleled the changes in detergent solubility of eNOS induced by stimuli which mostly elicit an apparent calcium-independent activation. The present findings suggest that VEGF stimulation promotes the formation of a stabilized multiprotein complex containing eNOS, hsp90, and Akt. It should be noted that when CHAPS or a Triton/molybdate mixture were used instead of octyl glucoside to lyse the cells and perform the immunoprecipitation, a low but significant amount of hsp90 associated to eNOS was consistently observed in the absence of any VEGF stimulation (not shown). Moreover, ex-periments in COS cells revealed that hsp90 also stimulates eNOS activation independently of Akt phosphorylation on ser-ine 1177 (see FIG. 12). It is postulated that a multicomplex including eNOS, caveolin, and hsp90 does exist and that local changes in calcium induces, in fact, the re-organization of the different partners within the complex and the recruitment of other players such as Akt.

Although, chronologically, the disruption of the caveolin/eNOS interaction precedes the hsp90/Akt recruitment, and [Ca²⁺]i chelation blocks both phenomena, it cannot be concluded that only the pool of eNOS initially bound to caveolin participates in the phosphorylation-mediated activation. The extent of eNOS associated to caveolin seems to vary according to the EC type and the state of cell confluence. However, an increase in the abundance of caveolin (native protein or peptido-mimetics) resulted in a marked decline in agonist-stimulated NO generation. Altogether, these observations suggest that caveolae could be a critical pool of activable eNOS in native cells. Further studies need to be performed to elucidate whether Akt selectively phosphorylates caveolar or intracellular pools of eNOS.

In conclusion, it was found that the binding of hsp90 is necessary for the recruitment of Akt to Ca²⁺/CaM-activated eNOS, thereby leading to the enzyme phosphorylation and the long-lasting NO release even in conditions of low [Ca²⁺]i Present findings also revealed that the binding of CaM to eNOS, as evoked by the initial Ca²⁺ transient, is obligatory to promote the long-term NO release at submaximal levels of [Ca²⁺]i. Thus, the so-called “calcium-independent” activation of eNOS appears misleading and it is possible that the term “phosphorylation-dependent eNOS activation” should be preferred at least in the context of VEGF stimulation. More generally, the identification of hsp90 as a key player in the eNOS phosphorylation pathway in EC (but also in myocytes, see FIG. 11E) opens new perspectives in the pharmacological regulation of NO-mediated pathways such as vasorelaxation and angiogenesis but also understanding of the physiopathology of diseases associated with alterations in hsp90 abundance such as hypoxia.

1. Pharmaceutical composition for modulating angiogenesis comprising a therapeutically effective amount of an angiogenesis modulating compound and a pharmaceutically acceptable excipient, wherein said modulating compound is a recombinant caveolin-1, a nucleic acid encoding the partial or total amino acid sequence of caveolin-1, or a pharmacologically acceptable derivative thereof or an analogue thereof.

2. Pharmaceutical composition for modulating angiogenesis comprising a therapeutically effective amount of an angiogenesis modulating compound and a pharmaceutically acceptable excipient, wherein said modulating compound is a recombinant caveolin-1 or a pharmacologically acceptable derivative thereof.

3. Pharmaceutical composition for modulating angiogenesis comprising a therapeutically effective amount of an angiogenesis modulating compound and a pharmaceutically acceptable excipient, wherein said modulating compound is a nucleic acid encoding the partial or total amino acid sequence of caveolin-1, or an analogue thereof.

4. Pharmaceutical composition of claim 3, whereby said nucleic acid is comprised in a recombinant expression vector.

5. Pharmaceutical composition for modulating angiogenesis comprising a therapeutically effective amount of an angiogenesis modulating compound and a pharmaceutically acceptable excipient, wherein said modulating compound is a nucleic acid modulating the expression of caveolin-1 thereby interfering with promoter, silencer or activator molecules or cites influencing the caveolin-1 mRNA population.

6. Pharmaceutical composition for modulating angiogenesis comprising a therapeutically effective amount of an angiogenesis modulating compound and a pharmaceutically acceptable excipient, wherein said modulating compound is influencing the translation or stability of the caveolin-1 mRNA.

7. Pharmaceutical composition of claim 6, whereby said compound is an antisense nucleic acid hybridising with a sequence encoding the partial or total amino acid sequence of caveolin-1.

8. Pharmaceutical composition of claim 7, whereby said antisense nucleic acid is comprised in a recombinant expression vector.

9. Pharmaceutical composition of claim 7 or 8, whereby said antisense nucleic acid is SEQ ID NO 5.

10. Pharmaceutical composition for modulating angiogenesis comprising a therapeutically effective amount of an angiogenesis modulating compound and a pharmaceutically acceptable excipient, wherein said modulating compound is a agonist or an antagonist of caveolin-1 thereby influencing the caveolin-1 activity.

11. Pharmaceutical composition for modulating angiogenesis comprising a therapeutically effective amount of an angiogenesis modulating compound and a pharmaceutically acceptable excipient, wherein said modulating compound is a competitive inhibitor of caveolin-1 by inhibiting the binding of the binding partners.

12. Pharmaceutical composition of claim 11, whereby said inhibitor is a scavenging or trapping molecule.

13. Pharmaceutical composition of claim 12, whereby said scavenging or trapping compound traps endogenous caveolin-1.

14. Pharmaceutical composition of claim 13, whereby said compound comprises an amino acid sequence pattern as described in SEQ ID NO 4, preferably comprises an amino acid sequence as described in SEQ ID NO 6 to SEQ ID NO 86.

15. Pharmaceutical composition of claim 12, whereby said scavenging/trapping compound traps endogenous eNOS mimicking the caveolin-1 molecule.

16. Pharmaceutical composition of claim 15, whereby said compound comprises SEQ ID NO 2 or SEQ ID NO 3 or portions thereof.

17. Pharmaceutical composition for modulating angiogenesis comprising a therapeutically effective amount of an angiogenesis modulating compound and a pharmaceutically acceptable excipient, wherein said modulating compound is a recombinant hsp90, a nucleic acid encoding the partial or total amino acid sequence of hsp90 or a pharmacologically acceptable derivative thereof or an analogue thereof.

18. Pharmaceutical composition for modulating angiogenesis comprising a therapeutically effective amount of an angiogenesis modulating compound and a pharmaceutically acceptable excipient, wherein said modulating compound is a recombinant hsp90, or a pharmacologically acceptable derivative thereof.

19. Pharmaceutical composition for modulating angiogenesis comprising a therapeutically effective amount of an angiogenesis modulating compound and a pharmaceutically acceptable excipient, wherein said modulating compound is a nucleic acid encoding the partial or total amino acid sequence of hsp90 or an analogue thereof.

20. Pharmaceutical composition of claim 19, whereby said nucleic acid is comprised in a recombinant expression vector.

21. Pharmaceutical composition for modulating angiogenesis comprising a therapeutically effective amount of an angiogenesis modulating compound and a pharmaceutically acceptable excipient, wherein said modulating compound is a nucleic acid modulating the expression of hsp90 thereby interfering with promoter/silencer/activator molecules/cites influencing the hsp90 mRNA population.

22. Pharmaceutical composition for modulating angiogenesis comprising a therapeutically effective amount of an angiogenesis modulating compound and a pharmaceutically acceptable excipient, wherein said modulating compound is influencing the translation or stability of the hsp90 mRNA.

23. Pharmaceutical composition of claim 22, whereby said compound is an antisense nucleic acid hybridising with a sequence encoding the partial or total amino acid sequence of hsp90.

24. Pharmaceutical composition of claim 23, whereby said antisense nucleic acid is comprised in a recombinant expression vector.

25. Pharmaceutical composition for modulating angiogenesis comprising a therapeutically effective amount of an angiogenesis modulating compound and a pharmaceutically acceptable excipient, wherein said modulating compound is a agonist or an antagonist of hsp90 thereby influencing the hsp90 activity.

26. Pharmaceutical composition for modulating angiogenesis comprising a therapeutically effective amount of an angiogenesis modulating compound and a pharmaceutically acceptable excipient, wherein said modulating compound is a competitive inhibitor of hsp90 by inhibiting the binding of the binding partners.

27. Pharmaceutical composition of claim 26, whereby said inhibitor is a scavenging or a trapping molecule.

28. Pharmaceutical composition for modulating angiogenesis comprising a therapeutically effective amount of an angiogenesis modulating compound and a pharmaceutically acceptable excipient, wherein said modulating compound is a recombinant Akt, a nucleic acid encoding the partial or total amino acid sequence of Akt or a pharmacologically acceptable derivative thereof or an analogue thereof.

29. Pharmaceutical composition for modulating angiogenesis comprising a therapeutically effective amount of an angiogenesis modulating compound and a pharmaceutically acceptable excipient, wherein said modulating compound is a recombinant Akt, or a pharmacologically acceptable derivative thereof.

30. Pharmaceutical composition for modulating angiogenesis comprising a therapeutically effective amount of an angiogenesis modulating compound and a pharmaceutically acceptable excipient, wherein said modulating compound is a nucleic acid encoding the partial or total amino acid sequence of Akt or an analogue thereof.

31. Pharmaceutical composition of claim 30, whereby said nucleic acid is comprised in a recombinant expression vector.

32. Pharmaceutical composition for modulating angiogenesis comprising a therapeutically effective amount of an angiogenesis modulating compound and a pharmaceutically acceptable excipient, wherein said modulating compound is a nucleic acid modulating the expression of Akt thereby interfering with promoter, silencer or activator molecules or cites influencing the Akt mRNA population.

33. Pharmaceutical composition for modulating angiogenesis comprising a therapeutically effective amount of an angiogenesis modulating compound and a pharmaceutically acceptable excipient, wherein said modulating compound is influencing the translation or stability of the Akt mRNA.

34. Pharmaceutical composition of claim 33, whereby said compound is an antisense nucleic acid hybridising with a sequence encoding the partial or total amino acid sequence of Akt.

35. Pharmaceutical composition of claim 34, whereby said antisense nucleic acid is comprised in a recombinant expression vector.

36. Pharmaceutical composition for modulating angiogenesis comprising a therapeutically effective amount of an angiogenesis modulating compound and a pharmaceutically acceptable excipient, wherein said modulating compound is a agonist or an antagonist of Akt thereby influencing the Akt activity.

37. Pharmaceutical composition for modulating angiogenesis comprising a therapeutically effective amount of an angiogenesis modulating compound and a pharmaceutically acceptable excipient, wherein said modulating compound is a competitive inhibitor of Akt by inhibiting the binding of the binding partners.

38. Pharmaceutical composition of claim 37, whereby said inhibitor is a scavenging or a trapping molecule.

39. A composition containing a statin and hsp90 and/or Akt or a pharmacologically acceptable derivative thereof as a combined preparation for simultaneous, separate or sequential use in a therapy in an angiogenesis-related disease.

40. A composition comprising a growth factor and hsp90 and/or Akt or a pharmacologically acceptable derivative thereof as a combined preparation for simultaneous, separate or sequential use in a therapy in an angiogenesis-related disease.

41. Use of hsp90 and/or Akt, or a pharmacologically acceptable derivative thereof, for the manufacturing of a medicament for the modulation of proangiogenic actions of statins and/or growth factors.

42. Method of modulating angiogenesis comprising the step of trapping endogenous caveolin-1 by a trapping compound thereby preventing its binding to the endothelial isoform nitric oxide synthase (eNOS).

43. Method according to claim 42, wherein said trapping compound comprises an amino acid sequence pattern as described in SEQ ID NO 4, preferably comprising the amino acid sequence pattern as described in SEQ ID NO 6 to SEQ ID NO 86.

44. A method of modulating angiogenesis comprising the step of trapping endogenous eNOS by a trapping compound whereby said trapping compound is mimicks caveolin-1.

45. Method according to claim 44, wherein said trapping compound comprises an amino acid sequence pattern as described in SEQ ID NO 2 or SEQ ID NO 3.

46. Method of modulating angiogenesis comprising the step of overexpressing or improving the activity of caveolin-1, hsp90, and/or Akt.

47. Method of modulating angiogenesis comprising the step of reducing the abundance and/or activity of caveolin-1, hsp90 and/or Akt.

48. Method for the treatment of angiogenesis related diseases comprising the step of administering a therapeutically effective amount of an angiogenesis modulating compound to an individual in need thereof, wherein said modulating compound is a recombinant of caveolin-1, a nucleic acid encoding the partial or total amino acid sequence of caveolin-1, or an analogue thereof or a pharmacologically acceptable derivative thereof, a compound modulating the expression of caveolin-1, an agonist or an antagonist or a competitive inhibitor of caveolin-1, a recombinant hsp90, a nucleic acid encoding the partial or total amino acid sequence of hsp90 or an analogue thereof or a pharmacologically acceptable derivative thereof, a compound modulating the expression of hsp90, an agonist or an antagonist or a competitive inhibitor of hsp90, a recombinant of Akt, a nucleic acid encoding the partial or total amino acid sequence of Akt, or an analogue thereof or a pharmacologically acceptable derivative thereof, a compound modulating the expression of Akt, an agonist or an antagonist or a competitive inhibitor of Akt

49. Method according to claim 48 wherein said angiogenesis related disease is angiogenesis dependent tumour growth and metastatic diseases, ischemic heart and peripheral vascular diseases including cerebral diseases and wound healing.

47. A diagnostic kit for the testing of a compound or a composition for their ability to modulate angiogenesis comprising an angiogenesis modulating compound wherein said modulating compound is a recombinant of caveolin-1, a nucleic acid encoding the partial or total amino acid sequence of caveolin-1, or an analogue thereof or a pharmacologically acceptable derivative thereof, a compound modulating the expression of caveolin-1, an agonist or an antagonist or a competitive inhibitor of caveolin-1, a recombinant hsp90, a nucleic acid encoding the partial or total amino acid sequence of hsp90 or an analogue thereof or a pharmacologically acceptable derivative thereof, a compound modulating the expression of hsp90, an agonist or an antagonist or a competitive inhibitor of hsp90, a recombinant of Akt, a nucleic acid encoding the partial or total amino acid sequence of Akt, or an analogue thereof or a pharmacologically acceptable derivative thereof, a compound modulating the expression of Akt, an agonist or an antagonist or a competitive inhibitor of Akt
